(19) United States
(12) Patent Application Publication
(10) Pub. No.: US 2023/0220448 A1
(43) Pub. Date: Jul. 13, 2023

(54) NON-INVASIVE PRENATAL SAMPLE PREPARATION AND RELATED METHODS AND USES

(71) Applicant: Myriad Women's Health, Inc., South San Francisco, CA (US)

(72) Inventors: Dale Muzzey, South San Francisco, CA (US); Genevieve Gould, South San Francisco, CA (US)

(73) Assignee: Myriad Women's Health, Inc., South San Francisco, CA (US)

(21) Appl. No.: 18/095,386

(22) Filed: Jan. 10, 2023

Related U.S. Application Data
(60) Provisional application No. 63/357,915, filed on Jul. 1, 2022.
(60) Provisional application No. 63/298,593, filed on Jan. 11, 2022.

(51) Int. Cl. C12Q 1/6809; C12Q 1/6883

(52) U.S. Cl. CPC C12Q 1/6809; C12Q 1/6883; C12Q 2600/156

(57) ABSTRACT

The present disclosure relates to methods of preparing cell-free DNA samples from expecting mothers or pregnant women, and related methods of analysis of such samples.

### CROSS REFERENCE TO RELATED APPLICATIONS

The present application claims the benefit of priority to U.S. Provisional Application No. 63/298,593 filed Jan. 11, 2022, and U.S. Provisional Application No. 63/357,915 filed Jul. 1, 2022, and the entire contents of each application are incorporated herein by reference.

### FIELD

Described herein are methods of preparing samples from expecting mothers, and related methods of analysis of such samples.

### BACKGROUND

The following description of the background of the present technology is provided simply as an aid in understanding the present technology and is not admitted to describe or constitute prior art to the present technology.

Non-invasive pre-natal screening (NIPS) has become a routine component of healthcare for expecting mothers. NIPS can involve both screening for aneuploidy (e.g., Down syndrome and the like) and screening for other genetic abnormalities in the mother or fetus. Many such screens utilize cell-free DNA (cfDNA); however, utilization of cfDNA suffers from a number of challenges because only a small portion of the cfDNA in maternal plasma is derived from the fetus.

Additionally, pre-natal screening for certain inheritable conditions has traditionally required obtaining DNA samples from both a mother and a father. For example, a traditional approach for detecting aneuploidy and various genetic conditions required obtaining samples of genomic DNA (gDNA) from both mother and father of the fetus, as well as cfDNA from the mother. Thus, such testing required at least three samples, each of which may be processed and assessed in a different manner.

The present disclosure addresses those challenges by providing methods of selectively enriching the fetal fraction of a maternal sample, such that NIPS for both aneuploidy and other genetic variants/mutations can be performed in parallel with only a single maternal sample.

SUMMARY

The present disclosure is generally directed to novel sample preparations and parallel screens for aneuploidy and other genetic variations, such as pathogenic SNPs, INDELs, and single gene copy number variations, from a single sample. These compositions and processes improve non-invasive pre-natal screening (NIPS) by streamlining and simplifying the necessary analysis, utilizing fewer samples, and reducing background noise, all with less complexity and requiring less time compared to conventional pre-natal screening analysis.

In one aspect, the present disclosure provides method of preparing a biological sample with an enriched fetal fraction, comprising:
(a-1) obtaining a biological sample comprising cell-free DNA (cfDNA) from a pregnant woman;
(b-1) extracting cfDNA from the biological sample;
(c-1) preparing a library of cfDNA fragments to obtain a cfDNA library
(d-1) separating the cfDNA fragments in the cfDNA library by size to retain only cfDNA fragments that are less than about 150 nucleotides in length, about 155 nucleotides in length, about 160 nucleotides in lengths, about 165 nucleotides in length, about 170 nucleotides in length, about 175 nucleotides in length, about 180 nucleotides in length, about 185 nucleotides in length, about 190 nucleotides in length, about 195 nucleotides in length, or about 200 nucleotides in length;
(e-1) sequencing the retained cfDNA fragments to obtain a first sequence library;
(f-1) identify based on read-length length (i) sequences of cell-free fetal DNA (cffDNA) and (ii) sequences of cell-free maternal DNA (cfmDNA) that are present in at least two windows of the first sequence library; and
(g-1) isolating the sequences of cffDNA from each of the at least two windows of the sequence library, thereby obtaining at least two fetal fraction-enriched sequence libraries or
(a-2) obtaining a biological sample comprising cell-free DNA (cfDNA) from a pregnant woman;
(b-2) extracting cfDNA from the biological sample;
(c-2) separating cfDNA fragments in the extracted sample from (b-2) to retain only cfDNA fragments that are less than about 150 nucleotides in length, about 155 nucleotides in length, about 160 nucleotides in lengths, about 165 nucleotides in length, about 170 nucleotides in length, about 175 nucleotides in length, about 180 nucleotides in length, about 185 nucleotides in length, about 190 nucleotides in length, about 195 nucleotides in length, or about 200 nucleotides in length;
(d-2) preparing a cfDNA library from the separated cfDNA fragments from (c-2);
(e-2) sequencing the cfDNA library to obtain a first sequence library;
(f-2) identify based on read-length length (i) sequences of cell-free fetal DNA (cffDNA) and (ii) sequences of cell-free maternal DNA (cfmDNA) that are present in at least two windows of the first sequence library; and
(g-2) isolating the sequences of cffDNA from each of the at least two windows of the sequence library, thereby obtaining at least two fetal fraction-enriched sequence libraries.

In some embodiments, separating the cfDNA fragments enriches the fetal fraction in the biological sample by about 1.1 fold, about 1.2 fold, about 1.3 fold, about 1.4 fold, about 1.5 fold, about 1.6 fold, about 1.7 fold, about 1.8 fold, about 1.9 fold, or about 2.0 fold.

In some embodiments, isolating the sequences of cffDNA from the at least two windows of the first sequence library enriches the fetal fraction in the biological sample by about 1.1 fold, about 1.2 fold, about 1.3 fold, about 1.4 fold, about 1.5 fold, about 1.6 fold, about 1.7 fold, about 1.8 fold, about 1.9 fold, about 2.0 fold, about 2.1 fold, about 2.2 fold, about 2.3 fold, about 2.4 fold, about 2.5 fold, about 2.6 fold, about 2.7 fold, about 2.8 fold, about 2.9 fold, about 3.0 fold, about 3.1 fold, about 3.2 fold, about 3.3 fold, about 3.4 fold, or about 3.5 fold.

In some embodiments, separating the cfDNA fragments comprises electrophoresis.

In some embodiments, at least 3, at least 4, at least 5, at least 6, at least 7, at least 8, at least 9, or at least 10 windows of the first sequence library are assessed to identify and isolate cffDNA sequences, thereby obtaining, respectively, at least 3, at least 4, at least 5, at least 6, at least 7, at least 8, at least 9, or at least 10 fetal fraction-enriched sequence libraries.

In some embodiments, the methods may further comprise identifying and separating cffDNA from cfmDNA by comparing sequence reads of cffDNA and cfmDNA in the first sequence library to a reference genome, demultiplexing sequence reads from the first library, removing duplicate sequences from the first sequence library, or a combination thereof.

In some embodiments, the methods may further comprises assessing the at least two fetal fraction-enriched sequence libraries for the presence of one or more genetic mutation(s). In some embodiments, the one or more genetic mutation(s) cause at least one condition selected from 21-Hydroxylase Deficiency, ABCC8-Related Hyperinsulinism, ARSACS, Achondroplasia, Achromatopsia, Adenosine Monophosphate Deaminase 1, Agenesis of Corpus Callosum with Neuronopathy, Alkaptonuria, Alpha-1-Antitrypsin Deficiency, Alpha-Mannosidosis, Alpha-Sarcoglycanopathy, Alpha-Thalassemia, Alzheimers, Angiotensin II Receptor, Type I, Apolipoprotein E Genotyping, Argininosuccinicaciduria, Aspartylglycosaminuria, Ataxia with Vitamin E Deficiency, Ataxia-Telangiectasia, Autoimmune Polyendocrinopathy Syndrome Type 1, BRCA1 Hereditary Breast/Ovarian Cancer, BRCA2 Hereditary Breast/Ovarian Cancer, Bardet-Biedl Syndrome, Best Vitelliform Macular Dystrophy, Beta-Sarcoglycanopathy, Beta-Thalassemia, Biotinidase Deficiency, Blau Syndrome, Bloom Syndrome, CFTR-Related Disorders, CLN3-Related Neuronal Ceroid-Lipofuscinosis, CLNS-Related Neuronal Ceroid-Lipofuscinosis, CLN8-Related Neuronal Ceroid-Lipofuscinosis, Canavan Disease, Carnitine Palmitoyltransferase IA Deficiency, Carnitine Palmitoyltransferase II Deficiency, Cartilage-Hair Hypoplasia, Cerebral Cavernous Malformation, Choroideremia, Cohen Syndrome, Congenital Cataracts, Facial Dysmorphism, and Neuropathy, Congenital Disorder of Glycosylationla, Congenital Disorder of Glycosylation Ib, Congenital Finnish Nephrosis, Crohn Disease, Cystinosis, DFNA 9 (COCH), Diabetes and Hearing Loss, Early-Onset Primary Dystonia (DYTI), Epidermolysis Bullosa Junctional, Herlitz-Pearson Type, FANCC-Related Fanconi Anemia, FGFR1-Related Craniosynostosis, FGFR2-Related Craniosynostosis, FGFR3-Related Craniosynostosis, Factor V Leiden Thrombophilia, Factor V R2 Mutation Thrombophilia, Factor XI Deficiency, Factor XIII Deficiency, Familial Adenomatous Polyposis, Familial Dysautonomia, Familial Hypercholesterolemia Type B, Familial Mediterranean Fever, Free Sialic Acid Storage Disorders, Frontotemporal Dementia with Parkinsonism-17, Fumarase deficiency, GJB2-Related DFNA 3 Nonsyndromic Hearing Loss and Deafness, GJB2-Related DFNB 1 Nonsyndromic Hearing Loss and Deafness, GNE-Related Myopathies, Galactosemia, Gaucher Disease, Glucose-6-Phosphate Dehydrogenase Deficiency, Glutaricacidemia Type 1, Glycogen Storage Disease Type la, Glycogen Storage Disease Type Ib, Glycogen Storage Disease Type II, Glycogen Storage Disease Type III, Glycogen Storage Disease Type V, Gracile Syndrome, HFE-Associated Hereditary Hemochromatosis, Halder AIMS, Hemoglobin S Beta-Thalassemia, Hereditary Fructose Intolerance, Hereditary Pancreatitis, Hereditary Thymine-Uraciluria, Hexosaminidase A Deficiency, Hidrotic Ectodermal Dysplasia 2, Homocystinuria Caused by Cystathionine Beta-Synthase Deficiency, Hyperkalemic Periodic Paralysis Type 1, Hyperornithinemia-Hyperammonemia-Homocitrullinuria Syndrome, Hyperoxaluria, Primary, Type 1, Hyperoxaluria, Primary, Type 2, Hypochondroplasia, Hypokalemic Periodic Paralysis Type 1, Hypokalemic Periodic Paralysis Type 2, Hypophosphatasia, Infantile Myopathy and Lactic Acidosis (Fatal and Non-Fatal Forms), Isovaleric Acidemias, Krabbe Disease, LGMD2I, Leber Hereditary Optic Neuropathy, Leigh Syndrome, French-Canadian Type, Long Chain 3-Hydroxyacyl-CoA Dehydrogenase Deficiency, MELAS, MERRF, MTHFR Deficiency, MTHFR Thermolabile Variant, MTRNR1-Related Hearing Loss and Deafness, MTTS1-Related Hearing Loss and Deafness, MYH-Associated Polyposis, Maple Syrup Urine Disease Type 1A, Maple Syrup Urine Disease Type 1B, McCune-Albright Syndrome, Medium Chain Acyl-Coenzyme A Dehydrogenase Deficiency, Megalencephalic Leukoencephalopathy with Subcortical Cysts, Metachromatic Leukodystrophy, Mitochondrial Cardiomyopathy, Mitochondrial DNA-Associated Leigh Syndrome and NARP, Mucolipidosis IV, Mucopolysaccharidosis Type I, Mucopolysaccharidosis Type IIIA, Mucopolysaccharidosis Type VII, Multiple Endocrine Neoplasia Type 2, Muscle-Eye-Brain Disease, Nemaline Myopathy, Neurological phenotype, Niemann-Pick Disease Due to Sphingomyelinase Deficiency, Niemann-Pick Disease Type C1, Nijmegen Breakage Syndrome, PPT1-Related Neuronal Ceroid-Lipofuscinosis, PROP1-related pituitary hormone deficiency, Pallister-Hall Syndrome, Paramyotonia Congenita, Pendred Syndrome, Peroxisomal Bifunctional Enzyme Deficiency, Pervasive Developmental Disorders, Phenylalanine Hydroxylase Deficiency, Plasminogen Activator Inhibitor I, Polycystic Kidney Disease, Autosomal Recessive, Prothrombin G20210A Thrombophilia, Pseudovitamin D Deficiency Rickets, Pycnodysostosis, Retinitis Pigmentosa, Autosomal Recessive, Bothnia Type, Rett Syndrome, Rhizomelic Chondrodysplasia Punctata Type 1, Short Chain Acyl-CoA Dehydrogenase Deficiency, Shwachman-Diamond Syndrome, Sjogren-Larsson Syndrome, Smith-Lemli-Opitz Syndrome, Spastic Paraplegia 13, Sulfate Transporter-Related Osteochondrodysplasia, TFR2-Related Hereditary Hemochromatosis, TPP1-Related Neuronal Ceroid-Lipofuscinosis, Thanatophoric Dysplasia, Transthyretin Amyloidosis, Trifunctional Protein Deficiency, Tyrosine Hydroxylase-Deficient DRD, Tyrosinemia Type I, Wilson Disease, X-Linked Juvenile Retinoschisis, cystic fibrosis, spinal muscular atrophy (SMA), a hemoglobinopathy, and Zellweger Syndrome Spectrum.

In some embodiments, the methods may further comprise assessing the biological sample comprising cfDNA for the presence of an aneuploidy. In some embodiments, the aneuploidy is selected from a monosomy, a trisomy, a tetrasomy, a pentasomy, a microdeletion, a micoduplication, and mosaic versions of monosomy, trisomy, tetrasomy, and pentasomy.

In another aspect, the present disclosure provides methods of parallel detecting the presence or absence of aneuploidy and the presence or absence of at least one genetic variant in a single, maternal sample, comprising
(i) obtaining a biological sample from a pregnant woman, wherein the biological sample comprises cell-free DNA (cfDNA);
(ii) preparing a cfDNA library;
(iii) sequencing the cfDNA library to produce a sequence library; and
(iv) detecting the presence or absence of aneuploidy and the presence or absence of at least one genetic variant in the single, maternal sample;
wherein (a) the cfDNA library is enriched to increase a fetal fraction, (b) the sequence library is enriched to increase a fetal fraction, or (c) a combination thereof, such that the fetal fraction of the single maternal sample is increased at least 1.1. fold, at least 1.2 fold, at least 1.3 fold, at least 1.4 fold, or at least 1.5 fold prior to detecting the presence or absence of aneuploidy and the presence or absence of at least one genetic variant in the single, maternal sample.

In some embodiments, the biological sample is blood, serum, or plasma.

In some embodiments, the cfDNA library is enriched to increase the fetal fraction and the sequence library is enriched to increase the fetal fraction.

In some embodiments, enriching the fetal fraction of the cfDNA library comprises removing from the cfDNA library any DNA fragments that are greater than about 150 nucleotides in length, about 155 nucleotides in length, about 160 nucleotides in lengths, about 165 nucleotides in length, about 170 nucleotides in length, about 175 nucleotides in length, or about 180 nucleotides in length. In some embodiments, removing the DNA fragments from the cfDNA library comprises electrophoresis.

In some embodiments, enriching the fetal fraction of the sequence library comprises a read-length-based size exclusion of sequences in at least two windows of the sequence library, thereby obtaining at least two fetal fraction-enriched sequence libraries. In some embodiments, at least 3, at least 4, at least 5, at least 6, at least 7, at least 8, at least 9, or at least 10 windows of the first sequence library are assessed to identify and isolate cffDNA sequences, thereby obtaining, respectively, at least 3, at least 4, at least 5, at least 6, at least 7, at least 8, at least 9, or at least 10 fetal fraction-enriched sequence libraries. In some embodiments, the at least two windows of the sequence library are selected from (i) sequences that are 0-145 nucleotides, (ii) sequences that are 0-150 nucleotides, (iii) 0-155 nucleotides, (iv) 0-160 nucleotides, (v) 0-165 nucleotides, (vi) 0-168 nucleotides, (vii) 0-170 nucleotides, (viii) 0-175 nucleotides, (ix) 0-180 nucleotides, (x) 0-185 nucleotides, (xi) 0-190 nucleotides, (xii) 0-195 nucleotides, (xiii) 0-200 nucleotides, and (xiv) ungated.

In some embodiments, enriching the fetal fraction of the sequence library further comprises identifying and separating cffDNA from cfmDNA by comparing sequence reads of cffDNA and cfmDNA in the first sequence library to a reference genome, demultiplexing sequence reads from the first library, removing duplicate sequences from the first sequence library, or a combination thereof

In some embodiments, detecting the presence or absence of at least one genetic variant comprises determining in each of the at least two fetal fraction-enriched sequence libraries an allele balance for each allele in the sample that encodes the at least one genetic variant, and generating an allele balance trajectory for each allele based on the allele balance in each of the at least two fetal fraction-enriched sequence libraries, a depth trajectory based on the depth of the at least two fetal fraction-enriched sequence libraries, or a combination of an allele balance trajectory and a depth trajectory.

In some embodiments, detecting the presence or absence of aneuploidy comprises analyzing a sequence depth of at least one sequence corresponding to a chromosome of interest in the sequence library. In some embodiments, the sequence depth of the at least one sequence corresponding to the chromosome of interest is fit to a model of expected depth for the chromosome of interest. In some embodiments, the sequence depth is calculated with the formula:

$${d}_{p} = ( 1 - f ) {c}_{m} \frac{{d}_{b}}{2} + {fc}_{f} \frac{{d}_{b}}{2}$$

where:

dₚ is pregnancy depth

f is fetal fraction

cₘ is maternal copy number

d_{b} is background depth

c_{f} is fetal copy number.

In some embodiments, the sequence depth is normalized to control for GC-bias, sample background, hybridization probe capture, or a combination thereof

In some embodiments, the method comprises detecting the presence or absence of aneuploidy selected from a monosomy, a trisomy, a tetrasomy, a polysomy X, a polysomy Y, a microdeletion, a microduplication, a pentasomy, and a combination thereof.

In some embodiments, the at least one genetic variant is associated with a disease selected from 21-Hydroxylase Deficiency, ABCC8-Related Hyperinsulinism, ARSACS, Achondroplasia, Achromatopsia, Adenosine Monophosphate Deaminase 1, Agenesis of Corpus Callosum with Neuronopathy, Alkaptonuria, Alpha-1-Antitrypsin Deficiency, Alpha-Mannosidosis, Alpha-Sarcoglycanopathy, Alpha-Thalassemia, Alzheimers, Angiotensin II Receptor, Type I, Apolipoprotein E Genotyping, Argininosuccinicaciduria, Aspartylglycosaminuria, Ataxia with Vitamin E Deficiency, Ataxia-Telangiectasia, Autoimmune Polyendocrinopathy Syndrome Type 1, BRCA1 Hereditary Breast/Ovarian Cancer, BRCA2 Hereditary Breast/Ovarian Cancer, Bardet-Biedl Syndrome, Best Vitelliform Macular Dystrophy, Beta-Sarcoglycanopathy, Beta-Thalassemia, Biotinidase Deficiency, Blau Syndrome, Bloom Syndrome, CFTR-Related Disorders, CLN3-Related Neuronal Ceroid-Lipofuscinosis, CLN5-Related Neuronal Ceroid-Lipofuscinosis, CLN8-Related Neuronal Ceroid-Lipofuscinosis, Canavan Disease, Carnitine Palmitoyltransferase IA Deficiency, Carnitine Palmitoyltransferase II Deficiency, Cartilage-Hair Hypoplasia, Cerebral Cavernous Malformation, Choroideremia, Cohen Syndrome, Congenital Cataracts, Facial Dysmorphism, and Neuropathy, Congenital Disorder of Glycosylationla, Congenital Disorder of Glycosylation Ib, Congenital Finnish Nephrosis, Crohn Disease, Cystinosis, DFNA 9 (COCH), Diabetes and Hearing Loss, Early-Onset Primary Dystonia (DYTI), Epidermolysis Bullosa Junctional, Herlitz-Pearson Type, FANCC-Related Fanconi Anemia, FGFR1-Related Craniosynostosis, FGFR2-Related Craniosynostosis, FGFR3-Related Craniosynostosis, Factor V Leiden Thrombophilia, Factor V R2 Mutation Thrombophilia, Factor XI Deficiency, Factor XIII Deficiency, Familial Adenomatous Polyposis, Familial Dysautonomia, Familial Hypercholesterolemia Type B, Familial Mediterranean Fever, Free Sialic Acid Storage Disorders, Frontotemporal Dementia with Parkinsonism-17, Fumarase deficiency, GJB2-Related DFNA 3 Nonsyndromic Hearing Loss and Deafness, GJB2-Related DFNB 1 Nonsyndromic Hearing Loss and Deafness, GNE-Related Myopathies, Galactosemia, Gaucher Disease, Glucose-6-Phosphate Dehydrogenase Deficiency, Glutaricacidemia Type 1, Glycogen Storage Disease Type 1a, Glycogen Storage Disease Type Ib, Glycogen Storage Disease Type II, Glycogen Storage Disease Type III, Glycogen Storage Disease Type V, Gracile Syndrome, HFE-Associated Hereditary Hemochromatosis, Halder AIMS, Hemoglobin S Beta-Thalassemia, Hereditary Fructose Intolerance, Hereditary Pancreatitis, Hereditary Thymine-Uraciluria, Hexosaminidase A Deficiency, Hidrotic Ectodermal Dysplasia 2, Homocystinuria Caused by Cystathionine Beta-Synthase Deficiency, Hyperkalemic Periodic Paralysis Type 1, Hyperornithinemia-Hyperammonemia-Homocitrullinuria Syndrome, Hyperoxaluria, Primary, Type 1, Hyperoxaluria, Primary, Type 2, Hypochondroplasia, Hypokalemic Periodic Paralysis Type 1, Hypokalemic Periodic Paralysis Type 2, Hypophosphatasia, Infantile Myopathy and Lactic Acidosis (Fatal and Non-Fatal Forms), Isovaleric Acidemias, Krabbe Disease, LGMD2I, Leber Hereditary Optic Neuropathy, Leigh Syndrome, French-Canadian Type, Long Chain 3-Hydroxyacyl-CoA Dehydrogenase Deficiency, MELAS, MERRF, MTHFR Deficiency, MTHFR Thermolabile Variant, MTRNR1-Related Hearing Loss and Deafness, MTTS1-Related Hearing Loss and Deafness, MYH-Associated Polyposis, Maple Syrup Urine Disease Type 1A, Maple Syrup Urine Disease Type 1B, McCune-Albright Syndrome, Medium Chain Acyl-Coenzyme A Dehydrogenase Deficiency, Megalencephalic Leukoencephalopathy with Subcortical Cysts, Metachromatic Leukodystrophy, Mitochondrial Cardiomyopathy, Mitochondrial DNA-Associated Leigh Syndrome and NARP, Mucolipidosis IV, Mucopolysaccharidosis Type I, Mucopolysaccharidosis Type IIIA, Mucopolysaccharidosis Type VII, Multiple Endocrine Neoplasia Type 2, Muscle-Eye-Brain Disease, Nemaline Myopathy, Neurological phenotype, Niemann-Pick Disease Due to Sphingomyelinase Deficiency, Niemann-Pick Disease Type C1, Nijmegen Breakage Syndrome, PPT1-Related Neuronal Ceroid-Lipofuscinosis, PROP1-related pituitary hormone deficiency, Pallister-Hall Syndrome, Paramyotonia Congenita, Pendred Syndrome, Peroxisomal Bifunctional Enzyme Deficiency, Pervasive Developmental Disorders, Phenylalanine Hydroxylase Deficiency, Plasminogen Activator Inhibitor I, Polycystic Kidney Disease, Autosomal Recessive, Prothrombin G20210A Thrombophilia, Pseudovitamin D Deficiency Rickets, Pycnodysostosis, Retinitis Pigmentosa, Autosomal Recessive, Bothnia Type, Rett Syndrome, Rhizomelic Chondrodysplasia Punctata Type 1, Short Chain Acyl-CoA Dehydrogenase Deficiency, Shwachman-Diamond Syndrome, Sjogren-Larsson Syndrome, Smith-Lemli-Opitz Syndrome, Spastic Paraplegia 13, Sulfate Transporter-Related Osteochondrodysplasia, TFR2-Related Hereditary Hemochromatosis, TPP1-Related Neuronal Ceroid-Lipofuscinosis, Thanatophoric Dysplasia, Transthyretin Amyloidosis, Trifunctional Protein Deficiency, Tyrosine Hydroxylase-Deficient DRD, Tyrosinemia Type I, Wilson Disease, X-Linked Juvenile Retinoschisis, cystic fibrosis, spinal muscular atrophy (SMA), a hemoglobinopathy, and Zellweger Syndrome Spectrum.

In another aspect, the present disclosure provides methods of enriching a biological sample for cell-free fetal DNA (cffDNA), comprising obtaining a biological sample comprising cell-free DNA (cfDNA) from a pregnant woman, wherein the cfDNA comprises cffDNA and cell-free maternal DNA (cfmDNA); extracting the cfDNA from the biological sample; and subjecting the extracted cfDNA to a size exclusion process, wherein the size exclusion process has a cutoff size of about 150 nucleotides in length, about 155 nucleotides in length, about 160 nucleotides in lengths, about 165 nucleotides in length, about 170 nucleotides in length, about 175 nucleotides in length, or about 180 nucleotides in length, thereby producing a sample enriched for cffDNA.

In another aspect, the present disclosure provides methods of in silico processing of cell-free DNA (cfDNA), comprising sequencing a cfDNA sample comprising cell-free fetal (cffDNA) and cell-free maternal DNA (cfmDNA) to prepare a sequence library; performing read-length-based analysis in which an allele balance for a nucleic acid sequence of interest is established in at least two windows of the sequence library; and establishing a trajectory based on the allele balance of the at least two windows.

In another aspect, the present disclosure provides methods of reducing background noise from superfluous genetic material in non-invasive pre-natal screening (NIPS), comprising
(i) obtaining a biological sample from a pregnant woman, wherein the biological sample comprises cell-free DNA (cfDNA); and
(ii) processing the cfDNA used for NIPS, wherein processing comprises enriching the biological sample for cell-free fetal DNA (cffDNA), in silico processing of the cfDNA, or a combination thereof.

In some embodiments, processing comprises both enriching the biological sample for cell-free fetal DNA (cffDNA) and in silico processing of the cfDNA.

In some embodiments, the enriching the biological sample for cell-free fetal DNA (cffDNA) comprises any one of the methods of enriching a biological sample for cell-free fetal DNA (cffDNA) disclosed herein.

In some embodiments, the in silico processing of the cfDNA comprises any one of the methods of in silico processing of cell-free DNA (cfDNA) disclosed herein.

In some embodiments, the method may further comprise normalization to control for GC-bias, sample background, hybridization probe capture, or a combination thereof.

The following detailed description is exemplary and explanatory, but it is not intended to be limiting.

### BRIEF DESCRIPTION OF THE DRAWINGS

FIG. 1 provides diagrams that compare conventional size exclusion techniques to the disclosed method of size exclusion, which is more permissive and retains more cffDNA.

FIG. 2 provides a visualization of the disclosed methods of in silico enrichment, which rely on a moving window analysis to closely observe changes in allele balance with changing amounts of fetal and maternal cfDNA.

FIG. 3 shows two ways of visualizing allele balance observed from the disclosed moving window analysis.

FIG. 4 shows an overview of an exemplary computational flow for one embodiment of the disclosed methods and systems.

FIG. 5 shows several visual representations of how depth calling can be used to establish the presence of an aneuploidy. The top panel compares a conventional karyotype to depth reads of chromosome 21 in a normal pregnancy and a pregnancy in which the fetus has trisomy 21. The middle panel represents the type of shift in depth that is expected when a trisomy is observed. The bottom panel shows the expected fit of four known copy number (CN) curves (e.g., CN=1, CN=2, CN=3, and CN=4) that represent various ploidies, which the shaded region indicating how the depth of a reading from a sample that includes a trisomy would fit within the expected fit curves.

FIG. 6 shows exemplary improvements in data plots that can be achieved by employing triple normalization controlling for variations caused by 1) GC bias, 2) sample background, and 3) hybridization probe capture.

FIG. 7 shows the fit of depth reads against expected fit curves for several chromosomes with different fit samples. The shaded region in each plot represents the depth of a given sample for the denoted chromosome. The fit curves, from left to right with each plot, are the expected fit for 1, 2, or 3 chromosomes for that fit model.

FIG. 8 shows a depth trajectory plot for a gene (SMN2) where the mother has one copy of the gene and fetus has zero.

### DETAILED DESCRIPTION

The sample preparations and methods disclosed herein are generally directed to novel processes of collecting a biological sample (e.g., blood or other DNA-containing sample) from a biological mother to then carry out screening, such as a parallel detection of aneuploidy and genetic mutations (e.g., a recessive surveillance procedure) through a non-invasive prenatal screen. That is, the present disclosure provides a single test (e.g., parallel) to discover two sets of detectable genetic conditions (e.g., aneuploidies and genetic variant screening) using samples from only one individual, namely a biological mother. Combining these two surveillance tests into a single test without involving the biological father provides efficiencies and convenience over conventional tests and methods, which often required a paternal sample and performed screening of aneuploidies and genetic variant screening separately. Moreover, the sample preparations may improve sensitivity, specificity, and minimize noise from superfluous genetic material that is unneeded for various causal genetic variant detection.

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless explicitly indicated otherwise, all specified embodiments, features, and terms intend to include both the recited embodiment, feature, or term and equivalents thereof.

### I. Definitions

As used herein, the singular forms “a,” “an,” and “the” designate both the singular and the plural, unless expressly stated to designate the singular only.

As used herein, the term “about” is to be understood as a relative term that encompasses both the stated numerical value and a range of +/−10%. For example, the phrase “about 10” should be understood as meaning both “10” and “9 to 11.”

Also as used herein, “and/or” refers to and encompasses any and all possible combinations of one or more of the associated listed items, as well as the lack of combinations when interpreted in the alternative (“or”).

As used herein, “optional” or “optionally” means that the subsequently described event or circumstance may or may not occur, and that the description includes instances where said event or circumstance occurs and instances where it does not.

As used herein, a “DNA-binding particle” refers to any conventional solid-phase material that interacts with, or that has been modified to interact with, a DNA fragment, such as a cfDNA fragment. The solid-phase phase material, for example, is any type of an insoluble, usually rigid material, matrix or stationary phase material that interacts with a DNA, either directly or indirectly, in a reaction solution. In certain example embodiments, the DNA-binding particle is a bead.

As used herein, a “bead” refers to a solid-phase particle of any convenient size, and can have an irregular or regular shape. In certain example embodiments, the surface of the bead is modified to bind DNA, either directly and/or indirectly. For example, the bead can include silanol groups, carboxylic groups, or other groups that facilitate the direct and/or interaction of the bead with DNA. In certain example embodiments, silica beads (and gels) can be functionalized by adding primary amines, thiols, sulfhydryls, propyl, octyl, as well as other derivatives to the hydroxyl group (silanol) attached to silica. The bead can fabricated from any number of known materials, including cellulose, cellulose derivatives, acrylic resins, glass, silica gels, polystyrene, gelatin, polyvinyl pyrrolidone, co-polymers of vinyl and acrylamide, polystyrene cross-linked with divinylbenzene, or the like, polyacrylamides, latex gels, polystyrene, dextran, rubber, silicon, plastics, nitrocellulose, natural sponges, silica gels, controlled pore glass (CPG), metals, cross-linked dextrans (e.g., Sephadex®), agarose gel (Sepharose®), and other solid phase bead supports known to those of skill in the art. In certain example embodiments, the beads can be packed together so as to form a column that can be used with conventional column chromatography.

As used herein, the term “genetic variant” when used in reference to a screening, call, or process described herein refers to an alteration from what is considered a non-pathogenic or wild-type gene sequence. Accordingly, the term “genetic variant” includes pathogenic single nucleotide polymorphisms (SNPs), insertions or deletions of bases within a subject's genome (INDELs), substitution mutations, single gene copy number variations, and the like. Additionally, it should be noted that the term “genetic variant” as used herein is distinct from aneuploidy and the term “genetic variant” does not relate to missing or extra chromosomes. Rather, the term “genetic variant” is to be understood as relating to features or alterations (pathogenic or otherwise) in a subject's genome sequence and not chromosomal abnormalities.

As used herein, the terms “cfDNA library” or “nucleic acid library” may be used interchangeably to refer to a collection of nucleic acids, e.g., a collection of cell free nucleic acids derived from a biological sample. In some embodiments, the cfDNA library or nucleic acid library is generated by amplifying the nucleic acid in a sample or otherwise preparing the library using PCR-free based methods. In some embodiments, the cfDNA library or nucleic acid library is generated by amplifying specific target fragments within a sample, as detailed below. In some embodiments, a portion or all of the nucleic acids in the cfDNA library or nucleic acid library comprise an adapter sequence. The adapter sequence can be located at one or both ends. The adapter sequence can be useful, e.g., for a sequencing method (e.g., an NGS method), for amplification, for reverse transcription, or for cloning into a vector.

The cfDNA library or nucleic acid library can comprise a collection of nucleic acid fragments, which may comprise a target nucleic acid sequence (e.g., a nucleic acid sequence in which a genetic variant associated with a disease can be detected), a reference nucleic acid sequence, or a combination thereof. In some embodiments, two or more cfDNA or nucleic acid libraries from the same subject can be combined.

As used herein, a “sequence library” is a collection of nucleic acid sequences that have been prepared by sequence a cfDNA library or nucleic acid library e.g., using massively parallel methods, such as next generation sequencing or NGS. NGS generally refers to sequencing methods that allow for massively parallel sequencing of clonally amplified and of single nucleic acid molecules during which a plurality, e.g., millions, of nucleic acid fragments from a single sample or from multiple different samples are sequenced in unison. Non-limiting examples of NGS include sequencing-by-synthesis, sequencing-by-ligation, real-time sequencing, and nanopore sequencing.

### II. Sample Preparations

Cell-free DNA (cfDNA) is a mixture of DNA which varies in properties (e.g., size, sequence, abundance) as well as tissue of origin (e.g., maternal vs. fetal). For example, cfDNA obtained from pregnant women contains DNA of both maternal and fetal origin. A primary driver of NIPS sensitivity when utilizing cfDNA in a given maternal plasma sample is the fetal fraction (FF). The fetal fraction comprises the portion of the total cell-free DNA that is from the fetus or derived from cell-free fetal DNA (cffDNA). For most samples, FF values are between 1% and 30%, but in many instances, the amount can be even lower.

The present disclosure provides sample preparations and methods of preparing samples from pregnant women (i.e., an expecting mother or biological mother) that can be used to improve sensitivity, specificity, and minimize noise when performing NIPS. In particular, the sample preparations may rely on physical processing of a cfDNA sample obtained from a pregnant woman, in silico processing of sequencing reads produced from a cfDNA sample obtained from a pregnant woman, or a combination thereof.

A. Physical Enrichment of the Fetal Fraction

Physical processing of a cfDNA sample (e.g., blood) obtained from a pregnant woman by methods of this disclosure can enrich the fetal fraction of a cfDNA sample by up to 3 times. In particular, the fetal fraction can be enriched in a sample by size selection using a size cut-off that retains most of the fetal cell-free DNA fragments and removes some of the large cell-free maternal DNA fragments. For example, a cut-off may be set to retain cfDNA fragments that are less than about 150 nucleotides in length, about 155 nucleotides in length, about 160 nucleotides in lengths, about 165 nucleotides in length, about 170 nucleotides in length, about 175 nucleotides in length, or about 180 nucleotides in length.

In some embodiments, the methods may be used to select and isolate fragments that are 75 nucleotides of less, 80 nucleotides of less, 85 nucleotides of less, 90 nucleotides of less, 95 nucleotides of less, 100 nucleotides of less, 105 nucleotides of less, 110 nucleotides of less, 115 nucleotides of less, 120 nucleotides of less, 125 nucleotides of less, 130 nucleotides of less, 135 nucleotides of less, 140 nucleotides of less, 145 nucleotides of less, 150 nucleotides of less, 155 nucleotides of less, 160 nucleotides of less, 165 nucleotides of less, 170 nucleotides of less, 175 nucleotides of less, 180 nucleotides of less, 195 nucleotides of less, 200 nucleotides of less, 205 nucleotides of less, 206 nucleotides of less, 210 nucleotides of less, 215 nucleotides of less, 220 nucleotides of less, 225 nucleotides of less, 230 nucleotides of less, 235 nucleotides of less, 240 nucleotides of less, 245 nucleotides of less, 250 nucleotides of less, 255 nucleotides of less, 260 nucleotides of less, 265 nucleotides of less, 270 nucleotides of less, 275 nucleotides of less, 280 nucleotides of less, 285 nucleotides of less, 290 nucleotides of less, 295 nucleotides of less, 300 nucleotides of less, 305 nucleotides of less, 310 nucleotides of less, 311 nucleotides of less, 315 nucleotides of less, 320 nucleotides of less, or 325 nucleotides of less. In some embodiments, the target size may be 125 nucleotides of less, 130 nucleotides of less, 135 nucleotides of less, 140 nucleotides of less, 145 nucleotides of less, 150 nucleotides of less, 155 nucleotides of less, 160 nucleotides of less, 165 nucleotides of less, 170 nucleotides of less, 175 nucleotides of less, 180 nucleotides of less, 195 nucleotides of less, or 200 nucleotides of less. Regardless of the precise cut-off or target size, the goal of the process is to retain cffDNA with little or no loss, and minimize or deplete cfmDNA.

This type of size-base exclusion can be performed using electrophoresis (e.g., gel electrophoresis or capillary electrophoresis) and other known methods, which may utilize, for example a DNA binding particle, such as a bead (e.g., an AMPURE™ bead). In one embodiment, nucleic acid electrophoretic separation followed by the recovery of the desired fragment lengths is used. Various known electrophoretic processes may be used for this purpose, but in one embodiment, the NIMBUS Select™ workstation with Ranger Technology™ for high throughput nucleic acid size selection may be used. Other strategies for fragment size selection include electrophoresis on agarose cassettes (BluePippin, Sage Science) following the manufacturer's instructions for “range” mode. Short fragments are eluted from the gel until the desired target size of the eluted DNA is obtained. Still other methods include, but are not limited to, solid support capture (e.g., affinity column), such as an antibody-coated spin column; synchronous (or non-synchronous) coefficient of drag alteration sizing (SCODA); solid phase reversible immobilization sizing (e.g., using carboxylated magnetic beads); affinity chromatography processes, or combinations of PCR amplification with varied lengths of amplicons and microchip separation.

The disclosed size-based exclusion methods may enrich the fetal fraction in a cfDNA sample by at least 1.1×, 1.2× 1.25×, 1.5×, 1.75×, 2×, 2.25×, 2.5×, 2.75×, 3×, 3.25×, 3.5×, 3.75×, 4×, 4.25×, 4.5×, 4.75×, 5×, 5.5×, 6×, 6.5×, 7×, 7.5×, 8×, 8.5×, 9×, 9.5×, 10×, 15×, 20×, 25× or more.

Thus, the present disclosure provides methods of size selection of cell-free fetal DNA (cffDNA), comprising subjecting a cell-free DNA (cfDNA) sample comprising cffDNA and cell-free maternal DNA (cfmDNA) to a size exclusion process in order to enrich a fetal fraction in a DNA sample obtained from a pregnant woman.

B. In Silico Enrichment of the Fetal Fraction

The present disclosure additionally provides in silico enrichment of a cfDNA sample (e.g., blood, plasma, serum) obtained from a pregnant woman, which are further able to enrich the fetal fraction of a cfDNA sample. In particular, the disclosed in silico enrichment comprises read-length-based size analysis. For the purposes of the present disclose, a “read-length-based size analysis” is an in silico process that establishes a trajectory from a range of windows that is applied to sequencing read data. The established trajectory is based on allele balances (ABs) observed across a set of FF levels. Thus, the FF levels are determined via in silico size selection from different windows, thus allowing for distinguishing between maternal and fetal DNA (cfmDNA and cffDNA, respectively). For example, a trajectory could show an AB of 55% at 10% FF, an AB of 60% at 15% FF, and an AB of 65% at 20% FF. This is an upward-sloping trajectory because the AB increases as FF increases. Both the slope and the offset (or intercept) of such a trajectory are useful. For instance, if cfmDNA are primarily selected by a given window, such that FF is as low as possible, the resulting AB mostly reflects the maternal genotype. As more FF is picked up by windows with smaller fragments, the deflection in AB is indicative of the fetal genotype. As a result, if the intercept is ˜50% (meaning that the mother is heterozygous for the variant), then a trajectory with negative slope suggests the fetus has not inherited a particular maternal variant.

Understanding the allele balance in the cfDNA sample improves the ability to focus on the desired sample fraction (e.g., FF for aneuploidy and genetic variant analysis, or maternal fraction for carrier analysis). In some embodiments, a moderate size selection in vitro (i.e., physical processing/size exclusion) followed by a size-based moving window analysis may provide the best results.

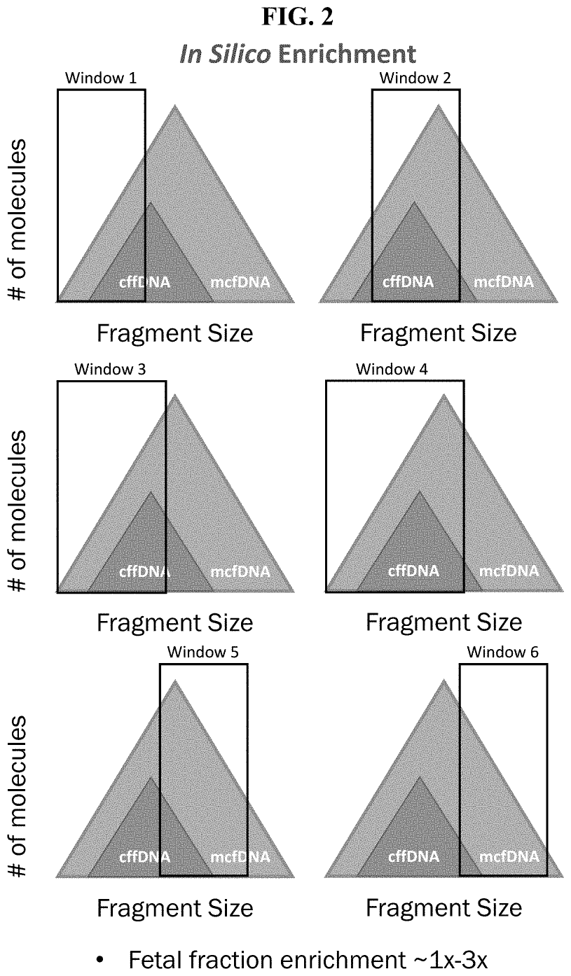

Once a sequence library has been prepared, the fetal fraction of the sequence library may be further processed or enriched using an in silico moving window analysis. For the purposes of the disclosed methods, a “window” is a selection or sub-section of the sequence library that includes a specific size range of sequences. For example, a “window” may encompass all of the sequences in the sequence library that are 0-145 nucleotides, 0-150 nucleotides, 0-155 nucleotides, 0-160 nucleotides, 0-165 nucleotides, 0-170 nucleotides, 0-175 nucleotides, 0-180 nucleotides, 0-185 nucleotides, 0-190 nucleotides, 0-195 nucleotides, 0-200 nucleotides, 0-205 nucleotide, 0-210 nucleotides, 0-215 nucleotides, 0-220 nucleotides, 0-225 nucleotides, 25-145 nucleotides, 25-150 nucleotides, 25-155 nucleotides, 25-160 nucleotides, 25-165 nucleotides, 25-170 nucleotides, 25-175 nucleotides, 25-180 nucleotides, 25-185 nucleotides, 25-190 nucleotides, 25-195 nucleotides, 25-200 nucleotides, 25-205 nucleotide, 25-210 nucleotides, 25-215 nucleotides, 25-220 nucleotides, 25-225 nucleotides, 50-145 nucleotides, 50-150 nucleotides, 50-155 nucleotides, 50-160 nucleotides, 50-165 nucleotides, 50-170 nucleotides, 50-175 nucleotides, 50-180 nucleotides, 50-185 nucleotides, 50-190 nucleotides, 50-195 nucleotides, 50-200 nucleotides, 50-205 nucleotide, 50-210 nucleotides, 50-215 nucleotides, 50-220 nucleotides, 50-225 nucleotides, 75-145 nucleotides, 75-150 nucleotides, 75-155 nucleotides, 75-160 nucleotides, 75-165 nucleotides, 75-170 nucleotides, 75-175 nucleotides, 75-180 nucleotides, 75-185 nucleotides, 75-190 nucleotides, 75-195 nucleotides, 75-200 nucleotides, 75-205 nucleotide, 75-210 nucleotides, 75-215 nucleotides, 75-220 nucleotides, 75-225 nucleotides, 100-145 nucleotides, 100-150 nucleotides, 100-155 nucleotides, 100-160 nucleotides, 100-165 nucleotides, 100-170 nucleotides, 100-175 nucleotides, 100-180 nucleotides, 100-185 nucleotides, 100-190 nucleotides, 100-195 nucleotides, 100-200 nucleotides, 100-205 nucleotide, 100-210 nucleotides, 100-215 nucleotides, 100-220 nucleotides, 100-225 nucleotides, or any ranges in between. A window can be considered “ungated” if a specific maximum and minimum are not set, and instead the window includes the entire sequence library. FIG. 2 shows an example in which the sequences in the sequence library are divided into four windows.

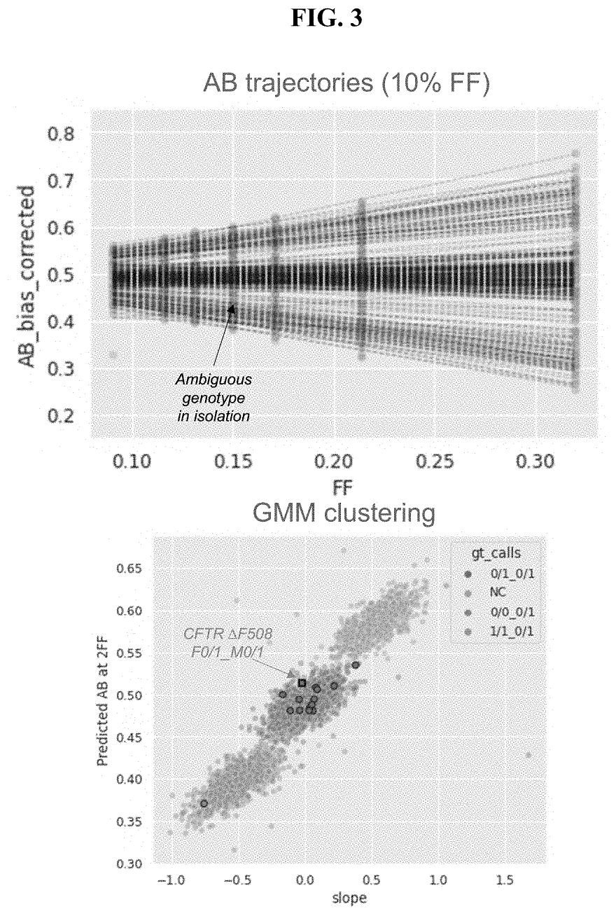

Thus, the disclosed methods of in silico enrichment can comprise a read-length-based size exclusion of sequences in at least two windows of the sequence library, thereby obtaining at least two fetal fraction-enriched sequence libraries. In some embodiments, 3, 4, 5, 6, 7, 8, 9, 10, or more windows may be assessed. In some embodiments, at least 5, at least 6 at least 7, or at least 8 windows may be assessed. In some embodiments, the windows are the same size (e.g., each window encompasses a set range of nucleotides, such as 0-100, 5-105, 10-110, etc.). In some embodiments, the windows are different sizes. For example, the size of each additional window may increase while the minimum remains the same (e.g., a set of windows with size cutoffs of 0-145, 0-150, 0-155, 0-160, 0-165, 0-170, etc.). Comparing the allele balance in each window allows for the calculation of an allele balance trajectory between the various fetal-fraction enriched sequence libraries. The trajectory is the change across the observed windows of the percentage of the allele balance for any given genetic sequence of interest. The allele balance trajectory can be calculated as a slope of the allele balance in each observed window, and it can be visualized in a number of ways, as shown in FIG. 3.

Further, the library of cfmDNA sequences can be enriched by focusing analysis between two fragment sizes, such as 100-200 nucleotides, 105-200 nucleotides, 110-200 nucleotides, 115-200 nucleotides, 120-200 nucleotides, 125-200 nucleotides, 130-200 nucleotides, 135-200 nucleotides, 140-200 nucleotides, 140-200 nucleotides, 145-200 nucleotides, 150-200 nucleotides, 155-200 nucleotides, 160-200 nucleotides, 165-200 nucleotides, 170-200 nucleotides, or 175-200 nucleotides or any size range in between. In some embodiments, the size range selected for enrichment may be about 155 to about 200 nucleotides.

In some embodiments, the at least two windows of the sequence library are selected from (i) sequences that are 0-145 nucleotides, (ii) sequences that are 0-150 nucleotides, (iii) 0-155 nucleotides, (iv) 0-160 nucleotides, (v) 0-165 nucleotides, (vi) 0-168 nucleotides, (vii) 0-170 nucleotides, (viii) 0-175 nucleotides, (ix) 0-180 nucleotides, (x) 0-185 nucleotides, (xi) 0-190 nucleotides, (xii) 0-195 nucleotides, (xiii) 0-200 nucleotides, and (xiv) ungated. In some embodiments, at least 3, at least 4, at least 5, at least 6, at least 7, at least 8, at least 9, or at least 10 windows are selected from (i) sequences that are 0-145 nucleotides, (ii) sequences that are 0-150 nucleotides, (iii) 0-155 nucleotides, (iv) 0-160 nucleotides, (v) 0-165 nucleotides, (vi) 0-168 nucleotides, (vii) 0-170 nucleotides, (viii) 0-175 nucleotides, (ix) 0-180 nucleotides, (x) 0-185 nucleotides, (xi) 0-190 nucleotides, (xii) 0-195 nucleotides, (xiii) 0-200 nucleotides, and (xiv) ungated.

Enriching the fetal fraction of the sequence library in silico can also further comprise identifying and separating cffDNA from cfmDNA by comparing sequence reads of cffDNA and cfmDNA in the first sequence library to a reference genome, demultiplexing sequence reads from the first library, removing duplicate sequences from the first sequence library, or a combination thereof

For instance, sample preparation can include in silico binary alignment processing in which the collected DNA sample may be computationally reconstructed by using overlaps between short sequencing reads. The reconstruction of a genome can be facilitated if a reference genome is available to which the sequencing reads can be aligned. A sequence alignment tool can be used to map short reads stored in a file to the reference genome. Subsequently, depth and variant processing can be used to identify and isolate specific gene sequences to inform follow-on analyses, which may be directed to, for example, identification of specific aneuploidies and/or genetic variants. In this way, with only a limited amount of initially collected cfDNA, specific portions of the collected DNA may be delineated and assembled for use with specific assay detections.

The collected DNA sample may be computationally reconstructed by using overlaps between short sequencing reads. Thus, DNA samples may be delineated at a first pass using a demultiplexer (e.g., demux), which allows for the determination unique molecule identifiers that may be needed for assessment for specific screenings (e.g., carrier, prenatal, and the like). Unique molecular identifiers (UMIs), (sometimes called molecular barcodes (MBC)) are short sequences (e.g., tags) added to DNA fragments during sequencing library preparation protocols to identify the desired DNA molecule upon which a specific screen may be directed. These tags are added before any amplification and can be used to reduce errors and quantitative bias introduced by the amplification.

Once tagged, the specific tagged DNA sequences may be initially aligned using an alignment processing to delineate the desired DNA sequences from each other. Then a duplication reduction (e.g., “deduping”) can clean up any errant identification and/or misalignments, which may comprise retaining a consensus sequence of overlapping portions of paired end reads. Thereafter, a realignment process can be performed to produce a more robust delineation between desired and tagged DNA sequences.

Amplification may be used to isolate specific nucleic acid sequences that are of interest or desirable for subsequent screening. For example, in silico amplification can be accomplished using computational tools to calculate theoretical polymerase chain reaction (PCR) results using a given set of primers (probes) to amplify DNA sequences from a sequenced DNA sample. After amplification, the quality of the specific read sequences may be improved by removing (e.g., trimming) partial (e.g., incomplete) sequences that are at beginnings and ending of sequences. One exemplary, but non-limiting, method for accomplishing this is called Paired-End (PE) trimming, which can include two input files (for forward and reverse reads) and four output files (for forward paired, forward unpaired, reverse paired and reverse unpaired reads) to identify and remove partial sequences. The reconstruction of a useful DNA sample can be facilitated and stored in a ready to use file. Further, the file may be delineated into different bins regarding fragment length (in terms of number of nucleotides).

Specific gene sequences stored in the file may be identified and isolated to inform follow-on analyses directed to specific aneuploidies and/or causal genetic variants as part of a depth and variant processing. This file may be used during specific procedures to alleviate biasing in the initial collected sample. The foregoing in silico steps and computational preparations can optimize the DNA sample for specific DNA sequences for the specific goals of a given test or screen.

The disclosed in silico processing may enrich the fetal fraction in a cfDNA sample by at least 1.1×, 1.2× 1.25×, 1.5×, 1.75×, 2×, 2.25×, 2.5×, 2.75×, 3×, 3.25×, 3.5×, 3.75×, 4×, 4.25×, 4.5×, 5.75×, 5×, 5.5×, 6×, 6.5×, 7×, 7.5×, 8×, 8.5×, 9×, 9.5×, 10×, 15×, 20×, 25× or more.

Alternatively, if desirable, the disclosed in silico processing may also be used to enrich the maternal fraction of a sample by selecting for larger fragments. In some embodiments, the disclosed in silico processing may enrich the maternal fraction in a cfDNA sample by at least 1.1×, 1.2× 1.25×, 1.5×, 1.75×, 2×, 2.25×, 2.5×, 2.75×, 3×, 3.25×, 3.5×, 3.75×, 4×, 4.25×, 4.5×, 5.75×, 5×, 5.5×, 6×, 6.5×, 7×, 7.5×, 8×, 8.5×, 9×, 9.5×, 10×, 15×, 20×, 25× or more.

Thus, the present disclosure provides methods of in silico sorting and enrichment of cffDNA, comprising sequencing a cell-free DNA (cfDNA) sample comprising cffDNA and cell-free DNA maternal (cfmDNA), and performing read-length-based size analysis, wherein a size-based moving window is used to establish a trajectory based on allele balances between cfmDNA and cffDNAto elucidate a genotype for the cfmDNA or cffDNA in a given sample. In some embodiments, such methods may further comprise identifying and separating cffDNA from cfmDNA by comparing sequence reads of cffDNA and cfmDNA to a reference genome, demultiplexing the sequence reads, and removing duplicate sequences.

C. Combination of Physical Enrichment and In Silico Enrichment

The foregoing methods of sample preparation can be performed individually or in combination to enrich the fetal fraction of a given sample. Prior to either physical enrichment or in silico enrichment, total cfDNA may be isolated from a maternal sample (e.g., blood, plasma, serum) by conventional means. For example, total cfDNA can extracted from clarified plasma obtained from a sample using an APOSTLE™ Cell-Free DNA Extraction kit. Other known methods and commercially available kits for cfDNA extraction can also be used, including but not limited to, kits produced Molzym GmbH & Co KG (Bremen, DE), Qiagen (Hilden, DE), Macherey-Nagel (Düren, DE), Roche (Basel, CH), and Sigma (Deisenhofen, DE).

After physical enrichment and in silico enrichment, the fetal fraction may be 2%, 3%, 4%, 5%, 6%, 7%, 8%, 9%, 10%, 11%, 12%, 13%, 14%, 16%, 17%, 18%, 19%, 20%, 21%, 22%, 23%, 24%, 25%, 26%, 27%, 28%, 29%, 30%,31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95%, 99% or 100% of the DNA sample that is used for further testing, screening, or analysis. Additionally or alternatively, after physical enrichment and in silico enrichment, the fetal fraction may be about 5% to 100%, about 5% to about 95%, about 5% to about 90%, about 5% to about 85%, about 5% to about 80%, about 5% to about 75%, about 10% to 100%, about 10% to about 95%, about 10% to about 90%, about 10% to about 85%, about 10% to about 80%, about 10% to about 75%, about 15% to 100%, about 15% to about 95%, about 15% to about 90%, about 15% to about 85%, about 15% to about 80%, about 15% to about 75%, about 20% to 100%, about 20% to about 95%, about 20% to about 90%, about 20% to about 85%, about 20% to about 80%, about 20% to about 75%, about 25% to 100%, about 25% to about 95%, about 25% to about 90%, about 25% to about 85%, about 25% to about 80%, about 25% to about 75%, about 30% to 100%, about 30% to about 95%, about 30% to about 90%, about 30% to about 85%, about 30% to about 80%, about 30% to about 75%, about 35% to 100%, about 35% to about 95%, about 35% to about 90%, about 35% to about 85%, about 35% to about 80%, about 35% to about 75%, about 40% to 100%, about 40% to about 95%, about 40% to about 90%, about 40% to about 85%, about 40% to about 80%, about 40% to about 75%, about 45% to 100%, about 45% to about 95%, about 45% to about 90%, about 45% to about 85%, about 45% to about 80%, about 45% to about 75%, about 50% to 100%, about 50% to about 95%, about 50% to about 90%, about 50% to about 85%, about 50% to about 80%, and about 50% to about 75%.

Thus, the present disclosure provides methods of preparing a cell-free DNA sample with an enriched fetal fraction, comprising processing of a cfDNA sample using size exclusion to retain cell-free fetal DNA (cffDNA) and remove cell-free maternal DNA (cfmDNA), in silico processing to identify and isolate cffDNA from cfmDNA, or a combination thereof.

### III. Methods of Parallel Screening

The present disclosure provides methods of assessing or screening for aneuploidy and genetic variants in a fetus utilizing only a single biological sample (e.g., blood, plasma, serum) from the biological mother of the fetus. Conventionally, testing for aneuploidy and testing for genetic variants were performed separately and required multiple samples. Indeed, screening for certain conditions even required a biological sample to be obtained from the biological father as well. The disclosed methods overcome these issues and function to provide new and useful methods that improve conventional non-invasive pre-natal screening (NIPS).

The disclosed methods may comprise two parallel screens that utilize the same single sample of cfDNA from the biological mother: a first screen for detecting aneuploidies and a second screen for detecting genetic variants.

In the first screen, a specific subsection of the collected sample (e.g., a subsection of smaller cfDNA fragments) can be used for optimizing the fetal fraction to assess the presence or absence of an aneuploidy condition. The presence or absence of the aneuploidy can be established by determining trajectories that allow for distinguishing maternal and fetal DNA. In this way, the disclosed screens can concurrent assess fetal aneuploidy and maternal aneuploidy, which was not previously possible. The first screen may additionally or alternatively rely on sequencing depth to determine whether an aneuploidy is present or absent in a given sample.

In the second screen, a specific subsection of the collected sample (e.g., a subsection of smaller cfDNA fragments) can be used optimizing the fetal fraction and minimizing noise from superfluous genetic material. The subsection can then be used for detecting various genetic variants by, for example, establishing trajectories to delineate relevant sample material from superfluous sample material. In each screen, using an optimal swath of a genetic sample that includes an appropriate ratio of cell-free maternal DNA (cfmDNA) to cell-free fetal cffDNA allows for detection with reasonable certainty of the presence or absence of known aneuploidies and genetic variants without having to resort to tailoring individual focus of the parallel screening toward one approach or the other.

The methods may begin with collecting a sample from a biological mother, typically through a blood draw, though other biological samples are contemplated (e.g., plasma, serum, etc.). This sample comprises cell free DNA (cfDNA). cfDNA may include various DNA freely circulating, including circulating tumor DNA (ctDNA), cell-free mitochondrial DNA (cf mtDNA), cell-free maternal DNA (cfmDNA) and cell-free fetal DNA (cffDNA). As the subject is an expecting mother, a certain level of fetal DNA will also be present in the cfDNA sample. Further, a targeted DNA capture suited to specific gene sequences may also be performed. Thus, aspects of both cfDNA as well as targeted capture may be employed for the purposes of the disclosed methods.

In one aspect, the present disclosure provides methods of parallel detection of the presence or absence of aneuploidy and at least one genetic mutation in a single, maternal sample, comprising (i) obtaining a biological sample from a pregnant woman, wherein the biological sample comprises cell-free DNA (cfDNA); (ii) preparing a cfDNA library (e.g., by amplifying a target population of cfDNA fragments); (iii) sequencing the cfDNA library to prepare a sequence library; and (iv) detecting the presence or absence of aneuploidy and at least one genetic variant in the single, maternal sample;
wherein (a) the cfDNA library is enriched to increase a fetal fraction, (b) the sequence library is enriched to increase a fetal fraction, or (c) a combination thereof, such that the fetal fraction of the single maternal sample is increased at least 1.5 fold prior to detecting the presence or absence of aneuploidy and at least one genetic variant in the single, maternal sample. In some embodiments, both the cfDNA library is enriched to increase the fetal fraction and the sequence library in enriched to increase the fetal fraction. The following sections provide more detail regarding relevant processes for each form of enrichment.

(i). Biological Sample

For the purposes of the disclosed methods, the biological sample needs to contain cfDNA, including cffDNA. Examples of samples that may be obtained from a biological mother for use in the disclosed methods include, but are not limited to, blood, serum, and plasma.

In some embodiments, nucleic acid extraction will be performed prior to amplification of the cfDNA in the sample and preparation of the cfDNA library or cfDNA libraries. Various protocols for nucleic acid extraction may be used in the methods of the present technology. Examples of commercially available nucleic acid purification kits include Apostle MiniMax Kit, Molzym GmbH & Co KG (Bremen, DE), Qiagen (Hilden, DE), Macherey-Nagel (Düren, DE), Roche (Basel, CH) or Sigma (Deisenhofen, DE). Other systems for nucleic acid purification, which are based on the use of polystyrene beads etc., as support material may also be used. Automated DNA extraction platforms may also be used, such as the QIAsymphony®, Hamilton® automation, or a Biorobot®EZ1™ automated system.

(ii). cfDNA Library Preparation

cfDNA library preparation can be performed using known methods of amplification (e.g., an xGen Prism Library Prep kit (IDT™)) as well as PCR-free methods of library preparation, such as COLLIBRI™, NEBNEXT® and TRUSEQ™ kits produced by Illumina, the KAPA™ HyperPrep kit produced by Roche, and the MGIEasy kit produced by MG Tech.. Optionally, preparation of the cfDNAlibrary can include a step of end repair. cfDNA may comprise overhangs of other damage to the ends of a given nucleic acid sequence, and end repair can convert such damaged or sheared DNA into blunt-ended molecules that are more easily ligated to adaptors, tags, or barcodes. One or more ligation reactions can be implemented to attach adaptors to the nucleic acid sequences from the sample. The adaptors are used to both facilitate amplification by providing a uniform sequence to which primers can anneal, and to separate the sequences of interest. Adaptors may be a unique length (to allow separation and isolation via electrophoresis), a unique sequence, or comprise other features to aid in isolation of target nucleic acid sequences after amplification.

PCR-based methods are commonly used to generate an amplified library in advance of sequencing or analysis of a given nucleic acid sample; however, PCR is not required, and those skilled in the art will know of PCR-free methods of library preparation as well. Various PCR methods utilizing commercially available reagents and polymerases may be utilized for the nucleic acid amplification portion of library preparation (e.g., KAPA™ HiFi HotStart ReadyMix).

Using any of the approaches described herein or otherwise known to those skilled in the art, a cfDNA library can be prepared from a maternal sample. Optionally, the cfDNA library can be cleaned using known methods, such as isolation of the amplified fragments in the library using AMPURE beads or other similar methods that allow for the removal of salts, unwanted macromolecules, and other debris from the sample.

Prior to sequencing of the cfDNA library, the fetal fraction may be enriched as described herein. Additionally or alternatively, the fetal fraction may be enriched the maternal sample prior to preparation of the cfDNA library. Briefly, enriching the fetal fraction of the cfDNA library or maternal sample is a physical processing of the sample, which can comprise removing from the cfDNA library any DNA fragments that are greater than about 150 nucleotides in length, about 155 nucleotides in length, about 160 nucleotides in lengths, about 165 nucleotides in length, about 170 nucleotides in length, about 175 nucleotides in length, about 180 nucleotides in length about 185 nucleotides in length, about 190 nucleotides in length, about 195 nucleotides in length, or about 200 nucleotides in length.

This type of size-base exclusion can be performed using electrophoresis (e.g., gel electrophoresis or capillary electrophoresis) and other known methods, which may utilize, for example a DNA binding particle, such as a bead (e.g., an AMPURE™ bead). In one embodiment, nucleic acid electrophoretic separation followed by the recovery of the desired fragment lengths is used. Various known electrophoretic processes may be used for this purpose. For example, in one embodiment, the NIMBUS Select™ workstation with Ranger Technology™ for high throughput nucleic acid size selection may be used. In another embodiments, the BluePippin electrophoresis system may be used.

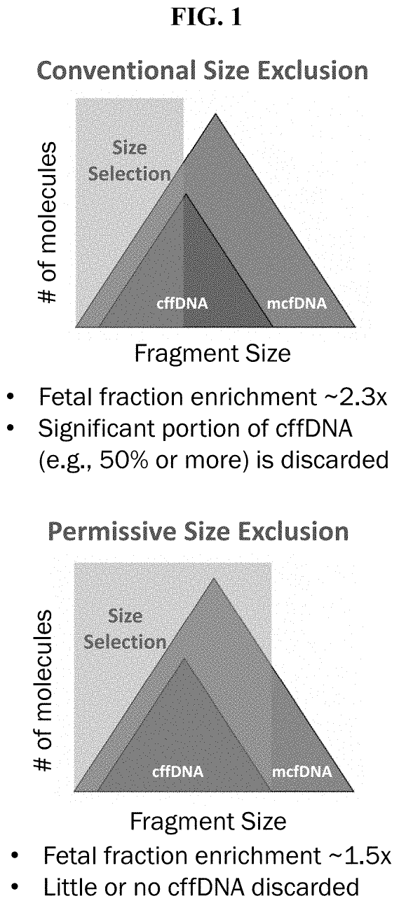

Prior methods of size-based exclusion have been used to enrich the fetal fraction of cfDNA libraries, but unlike those prior methods, the present inventors discovered that using a higher cutoff value can improve noise reduction when combined with further in silico selection, as described herein. Briefly, while not being bound by theory, noise may be reduced because of retention of a higher total number of cffDNA molecules via more permissive size selection. It was conventionally believed that using a lower cutoff value was superior because it excluded more maternal cfDNA. FIG. 1 shows a comparison of the disclosed size exclusion process compared to traditional approaches. As shown in FIG. 1, these more restrictive, traditional methods also discarded a not-insignificant amount of cffDNA. The disclosed approach of combining a more “permissive” size exclusion technique with a further in silico enrichment is thus an improvement that specifically addresses a critical problem in the field of pre-natal screening: enrichment of the fetal fraction without inadvertently or unnecessarily discarding cffDNA, which may be in preciously limited supply within a given sample.

The disclosed size-based exclusion methods may enrich the fetal fraction in a cfDNA sample by at least 1.1×, 1.2× 1.25×, 1.5×, 1.75×, 2×, 2.25×, 2.5×, 2.75×, 3×, 3.25×, 3.5×, 3.75×, 4×, 4.25×, 4.5×, 5.75×, 5×, 5.5×, 6×, 6.5×, 7×, 7.5×, 8×, 8.5×, 9×, 9.5×, 10×, 15×, 20×, 25× or more.

(iii). Sequencing the Nucleic Acid Library

The nucleic acid library, which may be enriched for fetal fraction, can be sequenced using known sequencing methods (e.g., NovaSeq sequencers and flowcells, Illumina sequencers, pyrosequencing, Reversible dye-terminator sequencing, SOLiD sequencing, Ion semiconductor sequencing, Helioscope single molecule sequencing, Ion Torrent™ (Life Technologies, Carlsbad, Calif.) amplicon sequencing system, 454™ GS FLX™ sequencing system, SMRT™ sequencing, etc.). In some embodiments, the cfDNA fragments in the nucleic acid library are sequenced from both ends (i.e., paired-end mode). In some embodiments, the cfDNA fragments in the nucleic acid library are sequenced are one end (i.e., single-end mode). In some embodiments, the cfDNA fragments in the nucleic acid library may be isolated or bound using a targeted capture method, such as hybrid capture. Sequencing from both ends of each fragment allows the fragment lengths to be determined. In some embodiments, the resulting sequences can be used to map the cfDNA fragments.

In some embodiments, the disclosed methods may utilize target capture methods to sequence only the particular fragments of interest. Fragments of interest may, for example, correspond to cfDNA that encodes a gene related to a genetic disease, condition, or trait (i.e., a genetic variant of interest) or cfDNA that corresponds to a particular chromosome.

Once the cfDNA fragments in the nucleic acid library have been sequenced, the fetal fraction of the sequence library may be further enriched using an in silico moving window analysis described herein. For the purposed of the disclosed methods, a “window” is a selection or sub-section of the sequence library that includes a specific size range of sequences. For example, a “window” may encompass all of the sequences in the sequence library that are 0-145 nucleotides, 0-150 nucleotides, 0-155 nucleotides, 0-160 nucleotides, 0-165 nucleotides, 0-170 nucleotides, 0-175 nucleotides, 0-180 nucleotides, 0-185 nucleotides, 0-190 nucleotides, 0-195 nucleotides, 0-200 nucleotides, 25-145 nucleotides, 25-150 nucleotides, 25-155 nucleotides, 25-160 nucleotides, 25-165 nucleotides, 25-170 nucleotides, 25-175 nucleotides, 25-180 nucleotides, 25-185 nucleotides, 25-190 nucleotides, 25-195 nucleotides, 25-200 nucleotides, 50-145 nucleotides, 50-150 nucleotides, 50-155 nucleotides, 50-160 nucleotides, 50-165 nucleotides, 50-170 nucleotides, 50-175 nucleotides, 50-180 nucleotides, 50-185 nucleotides, 50-190 nucleotides, 50-195 nucleotides, 50-200 nucleotides, 75-145 nucleotides, 75-150 nucleotides, 75-155 nucleotides, 75-160 nucleotides, 75-165 nucleotides, 75-170 nucleotides, 75-175 nucleotides, 75-180 nucleotides, 75-185 nucleotides, 75-190 nucleotides, 75-195 nucleotides, 75-200 nucleotides, 100-145 nucleotides, 100-150 nucleotides, 100-155 nucleotides, 100-160 nucleotides, 100-165 nucleotides, 100-170 nucleotides, 100-175 nucleotides, 100-180 nucleotides, 100-185 nucleotides, 100-190 nucleotides, 100-195 nucleotides, 100-200 nucleotides, or any ranges in between. In some embodiments, the disclosed methods may utilize two or more (e.g., 2, 3, 4, 5, 6, 7, 8, 9, or 10 or more) windows that encompass fragments in two or more (e.g., 2,3 ,4, 5, 6, 7, 8, 9, or 10 or more) size ranges selected from 0-145 nucleotides, 0-146 nucleotides, 0-147 nucleotides, 0-148 nucleotides, 0-149 nucleotides, 0-150 nucleotides, 0-151 nucleotides, 0-152 nucleotides, 0-153 nucleotides, 0-154 nucleotides, 0-155 nucleotides, 0-156 nucleotides, −157 nucleotides, 0-158 nucleotides, 0-159 nucleotides, 0-160 nucleotides, 0-161 nucleotides, 0-162 nucleotides, 0-163 nucleotides, 0-164 nucleotides, 0-165 nucleotides, 0-166 nucleotides, 0-167 nucleotides, 0-168 nucleotides, 0-169 nucleotides, 0-170 nucleotides, 0-171 nucleotides, 0-172 nucleotides, 0-173 nucleotides, 0-174 nucleotides, 0-175 nucleotides, 0-176 nucleotides, 0-177 nucleotides, 0-178 nucleotides, 0-179 nucleotides, 0-180 nucleotides, 0-181 nucleotides, 0-182 nucleotides, 0-183 nucleotides, 0-184 nucleotides, 0-185 nucleotides, 0-186 nucleotides, 0-187 nucleotides, 0-188 nucleotides, 0-189 nucleotides, 0-190 nucleotides, 0-191 nucleotides, 0-192 nucleotides, 0-193 nucleotides, 0-194 nucleotides, 0-195 nucleotides, 0-196 nucleotides, 0-197 nucleotides, 0-198 nucleotides, 0-199 nucleotides, 0-200 nucleotides, 5-145 nucleotides, 5-146 nucleotides, 5-147 nucleotides, 5-148 nucleotides, 5-149 nucleotides, 5-150 nucleotides, 5-151 nucleotides, 5-152 nucleotides, 5-153 nucleotides, 5-154 nucleotides, 5-155 nucleotides, 5-156 nucleotides, −157 nucleotides, 5-158 nucleotides, 5-159 nucleotides, 5-160 nucleotides, 5-161 nucleotides, 5-162 nucleotides, 5-163 nucleotides, 5-164 nucleotides, 5-165 nucleotides, 5-166 nucleotides, 5-167 nucleotides, 5-168 nucleotides, 5-169 nucleotides, 5-170 nucleotides, 5-171 nucleotides, 5-172 nucleotides, 5-173 nucleotides, 5-174 nucleotides, 5-175 nucleotides, 5-176 nucleotides, 5-177 nucleotides, 5-178 nucleotides, 5-179 nucleotides, 5-180 nucleotides, 5-181 nucleotides, 5-182 nucleotides, 5-183 nucleotides, 5-184 nucleotides, 5-185 nucleotides, 5-186 nucleotides, 5-187 nucleotides, 5-188 nucleotides, 5-189 nucleotides, 5-190 nucleotides, 5-191 nucleotides, 5-192 nucleotides, 5-193 nucleotides, 5-194 nucleotides, 5-195 nucleotides, 5-196 nucleotides, 5-197 nucleotides, 5-198 nucleotides, 5-199 nucleotides, 5-200 nucleotides, 10-145 nucleotides, 10-146 nucleotides, 10-147 nucleotides, 10-148 nucleotides, 10-149 nucleotides, 10-150 nucleotides, 10-151 nucleotides, 10-152 nucleotides, 10-153 nucleotides, 10-154 nucleotides, 10-155 nucleotides, 10-156 nucleotides, -157 nucleotides, 10-158 nucleotides, 10-159 nucleotides, 10-160 nucleotides, 10-161 nucleotides, 10-162 nucleotides, 10-163 nucleotides, 10-164 nucleotides, 10-165 nucleotides, 10-166 nucleotides, 10-167 nucleotides, 10-168 nucleotides, 10-169 nucleotides, 10-170 nucleotides, 10-171 nucleotides, 10-172 nucleotides, 10-173 nucleotides, 10-174 nucleotides, 10-175 nucleotides, 10-176 nucleotides, 10-177 nucleotides, 10-178 nucleotides, 10-179 nucleotides, 10-180 nucleotides, 10-181 nucleotides, 10-182 nucleotides, 10-183 nucleotides, 10-184 nucleotides, 10-185 nucleotides, 10-186 nucleotides, 10-187 nucleotides, 10-188 nucleotides, 10-189 nucleotides, 10-190 nucleotides, 10-191 nucleotides, 10-192 nucleotides, 10-193 nucleotides, 10-194 nucleotides, 10-195 nucleotides, 10-196 nucleotides, 10-197 nucleotides, 10-198 nucleotides, 10-199 nucleotides, 10-200 nucleotides, 15-145 nucleotides, 15-146 nucleotides, 15-147 nucleotides, 15-148 nucleotides, 15-149 nucleotides, 15-150 nucleotides, 15-151 nucleotides, 15-152 nucleotides, 15-153 nucleotides, 15-154 nucleotides, 15-155 nucleotides, 15-156 nucleotides, -157 nucleotides, 15-158 nucleotides, 15-159 nucleotides, 15-160 nucleotides, 15-161 nucleotides, 15-162 nucleotides, 15-163 nucleotides, 15-164 nucleotides, 15-165 nucleotides, 15-166 nucleotides, 15-167 nucleotides, 15-168 nucleotides, 15-169 nucleotides, 15-170 nucleotides, 15-171 nucleotides, 15-172 nucleotides, 15-173 nucleotides, 15-174 nucleotides, 15-175 nucleotides, 15-176 nucleotides, 15-177 nucleotides, 15-178 nucleotides, 15-179 nucleotides, 15-180 nucleotides, 15-181 nucleotides, 15-182 nucleotides, 15-183 nucleotides, 15-184 nucleotides, 15-185 nucleotides, 15-186 nucleotides, 15-187 nucleotides, 15-188 nucleotides, 15-189 nucleotides, 15-190 nucleotides, 15-191 nucleotides, 15-192 nucleotides, 15-193 nucleotides, 15-194 nucleotides, 15-195 nucleotides, 15-196 nucleotides, 15-197 nucleotides, 15-198 nucleotides, 15-199 nucleotides, 15-200 nucleotides, or any ranges in between. In some embodiments, the disclosed methods may utilize at least eight windows comprising size ranges including 0 to about 145 nucleotides, 0 to about 150 nucleotides, 0 to about 155 nucleotides, 0 to about 160 nucleotides, 0 to about 165 nucleotides, 0 to about 168 nucleotides, 0 to about 175 nucleotides, and 0 to about 190 nucleotides. In some embodiments, the disclosed methods may utilize eight windows comprising the size ranges 0-145 nucleotides, 0-150 nucleotides, 0-155 nucleotides, 0-160 nucleotides, 0-165 nucleotides, 0-168 nucleotides, 0-175 nucleotides, and 0-190 nucleotides.

In some embodiments, the disclosed methods may utilize two or more (e.g., 2, 3, 4, 5, 6, 7, 8, 9, or 10 or more) windows that encompass fragments in two or more (e.g., 2,3 ,4, 5, 6, 7, 8, 9, or 10 or more) size ranges selected from about 20 to about 145 nucleotides, about 20 to about 150 nucleotides, about 20 to about 155 nucleotides, about 20 to about 160 nucleotides, about 20 to about 165 nucleotides, about 20 to about 170 nucleotides, about 20 to about 175 nucleotides, about 20 to about 180 nucleotides, about 20 to about 185 nucleotides, about 20 to about 190 nucleotides, about 20 to about 195 nucleotides, about 20 to about 200 nucleotides, about 25 to about 145 nucleotides, about 25 to about 150 nucleotides, about 25 to about 155 nucleotides, about 25 to about 160 nucleotides, about 25 to about 165 nucleotides, about 25 to about 170 nucleotides, about 25 to about 175 nucleotides, about 25 to about 180 nucleotides, about 25 to about 185 nucleotides, about 25 to about 190 nucleotides, about 25 to about 195 nucleotides, about 25 to about 200 nucleotides, about 50 to about 145 nucleotides, about 50 to about 150 nucleotides, about 50 to about 155 nucleotides, about 50 to about 160 nucleotides, about 50 to about 165 nucleotides, about 50 to about 170 nucleotides, about 50 to about 175 nucleotides, about 50 to about 180 nucleotides, about 50 to about 185 nucleotides, about 50 to about 190 nucleotides, about 50 to about 195 nucleotides, about 50 to about 200 nucleotides, about 75 to about 145 nucleotides, about 75 to about 150 nucleotides, about 75 to about 155 nucleotides, about 75 to about 160 nucleotides, about 75 to about 165 nucleotides, about 75 to about 170 nucleotides, about 75 to about 175 nucleotides, about 75 to about 180 nucleotides, about 75 to about 185 nucleotides, about 75 to about 190 nucleotides, about 75 to about 195 nucleotides, about 75 to about 200 nucleotides, about 100 to about 145 nucleotides, about 100 to about 150 nucleotides, about 100 to about 155 nucleotides, about 100 to about 160 nucleotides, about 100 to about 165 nucleotides, about 100 to about 170 nucleotides, about 100 to about 175 nucleotides, about 100 to about 180 nucleotides, about 100 to about 185 nucleotides, about 100 to about 190 nucleotides, about 100 to about 195 nucleotides, about 100 to about 200 nucleotides, or any ranges in between.

For the purposes of the disclosed methods, the windows used for subsequent analysis and trajectory calculations can be different sizes (i.e., each window encompassing a different range of fragment sizes, such as 0-145, 0-150, 0-155, etc.) or the windows may be the same size (i.e., each window encompassing different fragments but across a set size range, such as 0-145, 5-150, 10-155, etc.). A window can be considered “ungated” is a specific maximum and minimum are not set, and instead the window includes the entire sequence library. FIG. 2 shows an example of how the sequences in the sequence library can be divided into six different windows.

As described above, enriching the fetal fraction of the sequence library is a form of in silico enrichment, which can comprise a read-length-based size exclusion of sequences in at least two windows of the sequence library, thereby obtaining at least two fetal fraction-enriched sequence libraries. Comparing the allele balance in each window allows for the calculation of an allele balance trajectory between the various fetal-fraction enriched sequence libraries. The allele balance trajectory is the change across the observed windows of the percentage of the allele balance for any given genetic sequence of interest. The allele balance trajectory can be calculated as a slope of the allele balance in each observed window, and it can be visualized in a number of ways, as shown in FIG. 3. For instance, the banding pattern in the top panel of FIG. 3 shows the divergence of the allele balance across multiple observed windows or the allele balance trajectory can be visualized as a Gaussian mixture model (GMM). It should be understood that each window (e.g., 0-145, 0-150, 0-155, etc.) will possess an associated fetal fraction that is distinct from the other windows, and this fetal fraction value can serve as the X-axis for a trajectory plot, as shown in FIG. 3 (top panel). In other words, the type of trajectory plot shown in FIG. 3 (top panel) provides a visualization of allele balance versus fetal fraction, wherein the points along the X axis (i.e., the fetal fraction axis) are provided by a selection of different windows.

Regardless of how the allele balance data is visualized, it can be utilized to identify heterozygous and homozygous mutations or markers of interest within the cfDNA sequence library. For example, the allele balance could be converted to a banding pattern plot in which the y-axis displays the percentage of cfDNA in a sample with a given allele for a particular gene or nucleic acid sequence of interest (e.g., 0%, 10%, 20%, 30%, 40%, 50%, 60%), and the x-axis displays different alternatives for the gene or nucleic acid of interest that correspond to the wild-type sequence or a mutation/variant associated with a particular disease, condition, or trait (e.g., different known mutations within the CFTR gene that are associated with cystic fibrosis).

By way of example, in a sample or window that has 20% fetal fraction may show a band at 10% on the y-axis corresponds to a fetus that is a carrier from the biological father's DNA (or, in some instances, it may represent a de novo mutation in the fetus). A band at 40% on the y-axis within this window corresponds to a fetus that is negative (i.e., homozygous reference) for the mutation/variant in the gene or sequence of interest. A band at 50% on the y-axis corresponds to a fetus that is a carrier from the biological mother's DNA or, if both the mother and the father carry the same mutation/variant (i.e., alt allele), it is possible that the fetus has the father's alt allele and the mother's reference allele. Thus, the band at 50% may indicate that the fetus and the mother each have one alt allele. A band at 60% on the y-axis corresponds to a fetus that is homozygous alt (i.e., the fetus is positive) for the mutation/variant in the gene or sequence of interest. As such, analyzing the allele balance across multiple windows of the sequence library (i.e., multiple fetal fraction-enriched sequence libraries) provides a new and useful way to establish the presence of absence of a genetic variant/mutation from a maternal sample comprising cfDNA without the need for any additional samples. Moreover, as a result of the enrichment provided by moving window analysis, noise and background are significantly reduced, which allows robust detection even in samples with vanishingly small amounts of cffDNA (e.g., <5% of total cfDNA). Additionally, it should be noted that the foregoing bands may shift or move, and they may not be precisely at 10%, 40%, 50%, and 60%, respectively, if the window or sample does not have 20% fetal fraction.

While at least two windows are needed in order to determine an allele balance trajectory, the number of windows that can be assessed for the purposes of the disclosed methods in not particularly limited and may include multiple additional windows. Thus, in some embodiments, at least 3, at least 4, at least 5, at least 6, at least 7, at least 8, at least 9, or at least 10 windows of the first sequence library are assessed, thereby obtaining, respectively, at least 3, at least 4, at least 5, at least 6, at least 7, at least 8, at least 9, or at least 10 fetal fraction-enriched sequence libraries from which cffDNA sequences can be identified and isolated. In some embodiments, the at least two windows of the sequence library are selected from (i) sequences that are 0-145 nucleotides, (ii) sequences that are 0-150 nucleotides, (iii) 0-155 nucleotides, (iv) 0-160 nucleotides, (v) 0-165 nucleotides, (vi) 0-170 nucleotides, (vii) 0-175 nucleotides, (viii) 0-180 nucleotides, (ix) 0-190 nucleotides, (x) 0-195 nucleotides, (xi) 0-200 nucleotides, and (xii) ungated. In some embodiments, at least 3, at least 4, at least 5, at least 6, at least 7, at least 8, at least 9, or at least 10 windows are selected from (i) sequences that are 0-145 nucleotides, (ii) sequences that are 0-150 nucleotides, (iii) 0-155 nucleotides, (iv) 0-160 nucleotides, (v) 0-165 nucleotides, (vi) 0-170 nucleotides, (vii) 0-175 nucleotides, (viii) 0-180 nucleotides, (ix) 0-190 nucleotides, (x) 0-195 nucleotides, (xi) 0-200 nucleotides, and (xii) ungated.

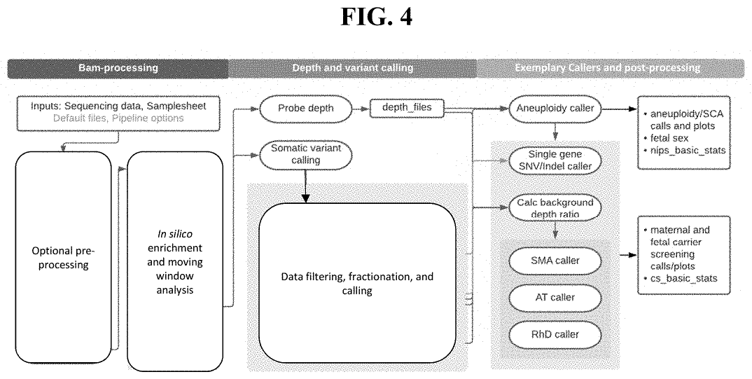

FIG. 4 provides an overview of one exemplary embodiment of a process of in silico enrichment of the fetal fraction within the sequence library and then using bioinformatics algorithms, which may also be referred to herein as “callers,” and post-processing to identify aneuploidies and genetic variants in parallel from a single sample.

A. Sample Processing and Computational Pipeline

In general, the sample processing steps for performing the disclosed methods of parallel assessment of aneuploidies and genetic variants can be performed as described in Section II (“Sample Preparations”) above. Further features are expanded on here.

The disclosed methods can comprise a computational pipeline that transforms the sequencing data from the sequence library into a useful output, which includes a determination of whether aneuploidy or any genetic variants are present in the cffDNA. Additional useful outputs that can optionally be provided include, but are not limited to, determination of fetal sex and other basic fetal statistics.

The computation pipeline may comprise Binary Alignment Map (BAM) processing in which a collected DNA sample may be computationally reconstructed using short sequencing reads. The reconstruction of a genome can be facilitated if a reference genome is available to which the sequencing reads can be aligned. A sequence alignment tool can be used to map short reads stored in a file to the reference genome. This generates a BAM file wherein specific gene sequences may be dealt with in the next step.

The computation pipeline may also comprise depth and variant processing, during which specific gene sequences may be identified and isolated to inform follow-on analyses directed to specific aneuploidies and/or genetic variants. Based on the amount of initial DNA collected, specific portions of the collected DNA may be delineated and, optionally, assembled for use with analysis and detection of specific sequences of interest. Once delineated at the depth and variant processing step, specific callers and post processing may be used to identify and assemble output information regarding aneuploidy, genetic variants, and any other outputs into a results report. The results are generally reported, delivered, or transmitted to the mother, the father, the physician overseeing the pregnancy (i.e., the mother's OBGYN), or a combination thereof.

The depth of the DNA sample in the BAM file can be assessed using specific bioinformatic algorithms (i.e., “calling procedures”; described below). The callers used can determine the presence or absence of both aneuploidy and genetic variants of interest. That is, these two goals can be accomplished together (e.g., in a parallel manner) using the same prepared and processed BAM file. Thus, aneuploidies may be detected using an aneuploidy caller program, while other genetic variants using a dedicated caller program can be run in parallel. Specific aspects of these computational steps are discussed in more detail below.

It should be understood that the present disclosures as described above can be implemented in the form of control logic using computer software in a modular or integrated manner. Based on the disclosure and teachings provided herein, a person of ordinary skill in the art will know and appreciate other ways and/or methods to implement the present disclosure using hardware and a combination of hardware and software.

Any of the software components, processes or functions described in this application may be implemented as software code to be executed by a processor using any suitable computer language such as, for example, Python, R, Assembly language Java, JavaScript, C, C++ or Perl using, for example, conventional or object-oriented techniques. The software code may be stored as a series of instructions, or commands on a computer readable medium, such as a random-access memory (RAM), a read only memory (ROM), a magnetic medium such as a hard-drive or a floppy disk, or an optical medium such as a CD-ROM. Any such computer readable medium may reside on or within a single computational apparatus and may be present on or within different computational apparatuses within a system or network.

B. Detection of Aneuploidy

For the purposes of this disclosure, aneuploidies that may be assessed or detected using the disclosed methods include, but are not limited to, monosomy (e.g., Turner syndrome), trisomy (e.g., Down syndrome, Edwards syndrome, Patau syndrome, trisomy 13, trisomy, 18, trisomy 21), tetrasomy, polysomy X and/or Y, microdeletions and micro duplications (such as Chromosome 22q11.2 deletion syndrome), and pentasomy.

The present disclosure provides systems and methods for detecting aneuploidies, either alone or in parallel with genetic variants/mutations of interest that rely on sequencing depth to determine whether an aneuploidy is present or absent in a given sample. For the purposes of this disclosure, “depth” is defined as the ratio of the number of reads obtained by sequencing that overlap with a site of interest to the size of the library or the average number of times each base is measured in the library.

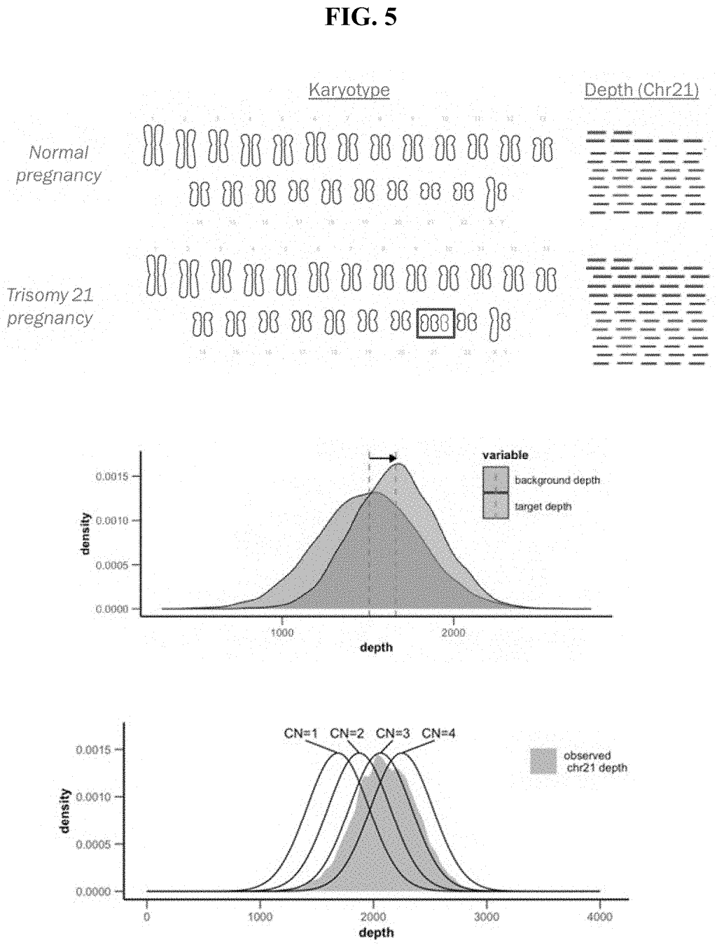

The observed depth in any given library that is prepared from a maternal cfDNA sample is a function of fetal fraction, maternal copy number, and fetal copy number. If an aneuploidy (e.g., trisomy) is present, the depth in target chromosome should be different from a sample with 23 chromosomes in a defined, predictable way. For instance, in a trisomy, the depth in a target chromosome (e.g., chromosome 21) will increase compared to the background. FIG. 5 illustrates the principles underlying this measure.

In general, when detecting aneuploidies (whether it is a fetal aneuploidy or a maternal aneuploidy) within a maternal sample that includes some fraction of fetal cfDNA (e.g., the fetal fraction), the presence of an aneuploidy can be identified based on a shift in a detectable aneuploid region or aneuploid chromosome in comparison to known non-aneuploidy regions or chromosomes. That is, depending on the actual fetal fraction, an analysis (e.g., Formula 1, below) of each fragment will yield a plottable result of cfDNA pregnancy depth against cfDNA density. This shift can be calculated statistically or visualized, as shown in the middle panel of FIG. 5, in which the background depth represents a comparator or aggregate of samples without an aneuploidy and the shifted target depth represents a sample that includes a trisomy, thus indicating the presence of the aneuploidy in the fetus (presuming that the expectant mother does not exhibit said aneuploidy). This deviation is detectable using normalized distribution curves and will be more pronounced as the fetal fraction of the sample is increased via the enrichment processes described herein.

In some embodiments, a depth calling plot (shown in the bottom panel of FIG. 5) can be used to visualize and quantify shifts. As shown in the bottom panel of FIG. 5 the depth of a given sample (i.e., the shaded area) may be determined to fit within one of four known copy number (CN) curves (e.g., CN=1, CN=2, CN=3, and CN=4. In this exemplary figure, the shaded distribution fits within the CN=3 curve, thus indicating the existence of an aneuploidy with three copies of a chromosome (i.e., a trisomy). Various processing steps may be employed to enhance distribution plot results and quell noise in the data during analysis.

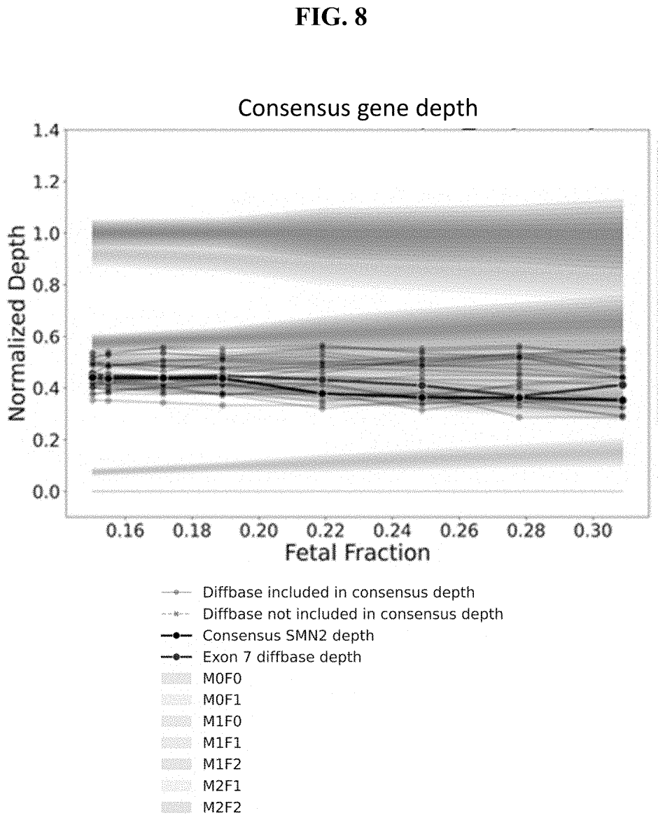

In some embodiments, detecting the presence or absence of an aneuploidy may comprise calculating a depth trajectory. The depth trajectory is the change across observed windows of the read depth for any given genetic sequence of interest. The depth trajectory can be calculated as a slope of the depth versus fetal fraction across observed window, and it can be visualized in a number of ways, as shown in FIG. 8. A depth trajectory that decreases while fetal fraction increases would indicate the fetus has less copies of the gene (or chromosome) than the mother. A depth trajectory that stays constant as fetal fraction increases would indicate that the fetus and mother have the same copy number of the gene (or chromosome). And a depth trajectory that increases as fetal fraction increases would indicate that the fetus has more copies of the gene (or chromosome) than the mother. While depth trajectories and useful in determining chromosome number for the purposes of detecting the presence or absence of an aneuploidy, it should be noted that depth trajectories may also be used to detect the presence or absence of certain genetic variants, such as copy number abnormalities.

During analysis of chromosome depth for any given sample, it may be necessary to account for and normalize GC-bias. GC-content (or guanine-cytosine content) is the percentage of nitrogenous bases on a DNA molecule that are either guanine or cytosine (from a possibility of four different ones, also including adenine and thymine). A high GC content can skew results and lead to high levels of noise. For example, in the context of FIG. 5C, increasing noise would broaden the width of the data bands and the corresponding copy-number hypotheses (black lines), and as these distributions get wider, it becomes more difficult to accurately interpret the true copy-number level. Correct normalization reduces variance in depth in high noise samples, thereby reducing effects of GC bias and improving aneuploidy calling.

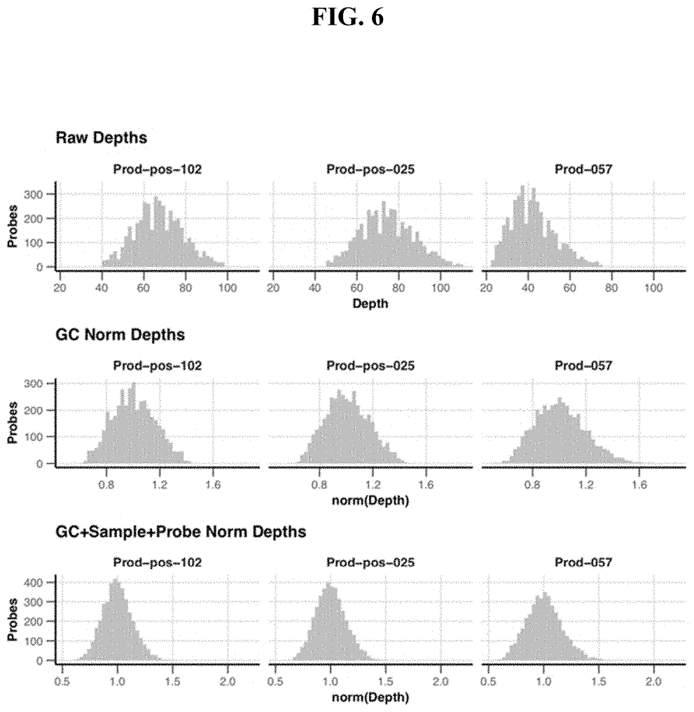

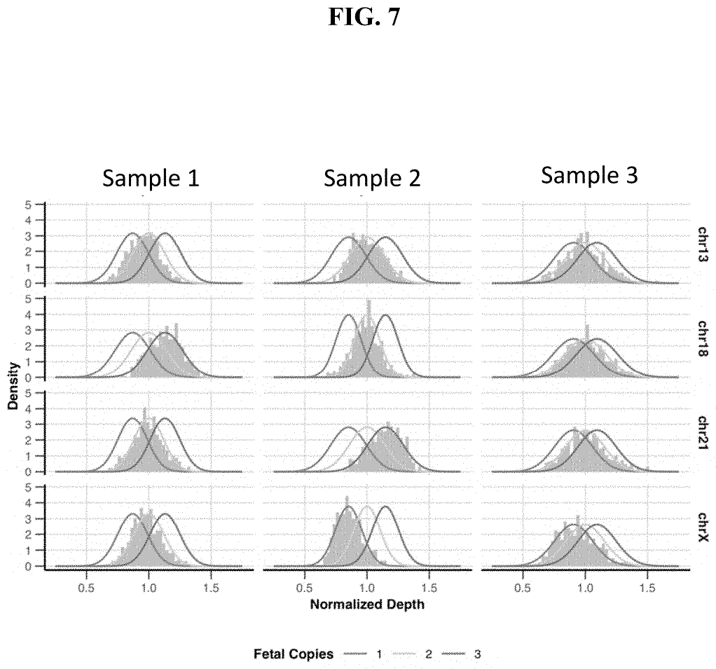

Further, triple normalization controlling for variations caused by 1) GC bias, 2) sample background, and 3) hybridization probe capture (when appropriate; i.e., in embodiments utilizing hybrid probes) may be employed across sampled data to improve the distribution plots of the sampled data as shown in FIG. 6. As provided in FIG. 6, a top set of distribution plots show raw depth data without any normalization, the middle set of distribution plots show improved distribution plots after GC bias normalization is employed, and the bottom set of distribution plots are even more improved after second (sample background) and third (hybridization probe capture) normalization data processing steps are accomplished. Thus, triple normalization controlling can improve the distribution plots of sampled data and may be useful in certain disclosed embodiments or for certain samples. Once normalized, these distribution plots may be compared to model expectations to derive conclusions about the presence or absence aneuploidies, as illustrated in FIG. 7.

FIG. 7 shows diagrams of normalized depths fit model expectations of incidence of aneuploidies that may be used to decipher assembled and, optionally, normalized sample distributions. Depths Fit models may be assembled using conventional known aneuploidy distribution for use in a comparison step to decipher whether the actual assembled and, optionally, normalized distributions match one or more of the assembled known models. As shown in FIG. 7, the normalized depth distributions, shown in grey may be set against known distribution curves that reflect 1, 2, or 3 copies (in that order, from left to right) for chromosomes 13, 18, 21, and X. The specific curve fits may be determined using maximum likelihood to select to most likely fetal copy number. As a maximum likelihood fit yields a match to specific call, a conclusion can be drawn with respect to the presence or absence of an aneuploidy within an analyzed sample.

Based on the predicted differences in depth that will be observed when an aneuploidy is present in a sample, an aneuploidy caller can be designed to select a set of maternal and fetal copy numbers that generates the highest likelihood of aneuploidy on a normal distribution. To this end, the following equation was developed for determining depth of a given aneuploidy:

$${d}_{p} = ( 1 - f ) {c}_{m} \frac{{d}_{b}}{2} + {fc}_{f} \frac{{d}_{b}}{2} \qquad [ Formula 1 ]$$

where:

dₚ is plasma depth

f is fetal fraction

cₘ is maternal copy number

d_{b} is background depth

c_{f} is fetal copy number

This caller was shown to be both highly sensitive and specific for detecting autosomal and sex chromosome aneuploidies, as well as fetal sex calls. The Examples, below, provide further detail regarding the performance of the aneuploidy caller.

After completion of a screening as disclosed herein, a physician may choose to administer further assessments, such as an Expanded Aneuploidy Analysis (EAA) that analyzes even more numbered chromosome pairs to provide additional insights into the health of the pregnancy. Accordingly, in some embodiments, the disclosed methods of determining the presence or absence of an aneuploidy may further comprise an EAA.

C. Detection of Genetic Variants

In general, the genetic variants (e.g., genetic mutations) that are detected as part of the disclosed methods are genetic variants, markers, or mutations that are associated with specific genetic or inheritable diseases, conditions, or traits. Genetic variants may include single nucleotide variations (SNVs), pathogenic or non-pathogenic single nucleotide polymorphisms (SNPs), insertions and deletions (indels), substitution mutations, or single gene copy number variants.

A genetic variant can be associated with more than one disease, condition, or trait. Genetic variants can manifest as variations in a polynucleotide, such as at least about 1, 2, 3, 4, 5, 6, 7, 8, 9, 10, 20, 30, 40, 50 or more sequence differences between a wild-type (i.e., non-mutated or unassociated with a disease or condition) gene or locus. Non-limiting examples of types of genetic variants that can be detected using the disclosed methods include, but are not limited to, single nucleotide polymorphisms (SNP), deletion/insertion polymorphisms (DIP), micro- copy number variants (CNV), short tandem repeats (STR), restriction fragment length polymorphisms (RFLP), single sequence repeats (SSR), variable number of tandem repeats (VNTR), randomly amplified polymorphic DNA (RAPD), amplified fragment length polymorphisms, retrotransposon-based insertion polymorphism, sequence specific amplified polymorphism, and heritable epigenetic modifications (for example, DNA methylation).

For the purposes of the disclosed methods, the presence or absence of at least 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1000 or more different genetic variants may be detected is a single assay and in parallel with a detection of the presence or absence of aneuploidy. In some embodiments, the methods may detect in parallel the presence or absence of genetic variants that are associated with at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 or more diseases, conditions, or traits.

In general, the presence of the types of genetic variants that are detected by the disclosed methods are associated with increased risk of having or developing the disease, condition, or trait by about, less than about, or more than about 1%, 5%, 10%, 15%, 20%, 25%, 30%, 40%, 50%, 60%, 70%, 80%, 90%, 100%, 200%, 300%, 400%, 500%, or more. In some embodiments, the presence of a genetic variant increases the risk of having or developing a disease, condition, or trait by about, less than about, or more than about 1-fold, 2-fold, 3-fold, 4-fold, 5-fold, 6-fold, 7-fold, 8-fold, 9-fold, 10-fold, 25-fold, 50-fold, 100-fold, 500-fold, 1000-fold, 10000-fold, or more. In some embodiments, the presence of a genetic variant increases the risk of having or developing a disease, condition, or trait by any statistically significant amount, such as an increase having a p-value of about or less than about 0.1, 0.05, 10⁻³, 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵, or smaller.

For the purposes of this disclosure, genetic diseases that may be assessed or detected by determining the presence or absence of a genetic variant include, but are not limited to, 21-Hydroxylase Deficiency, ABCC8-Related Hyperinsulinism, ARSACS, Achondroplasia, Achromatopsia, Adenosine Monophosphate Deaminase 1, Agenesis of Corpus Callosum with Neuronopathy, Alkaptonuria, Alpha-1-Antitrypsin Deficiency, Alpha-Mannosidosis, Alpha-Sarcoglycanopathy, Alpha-Thalassemia, Alzheimers, Angiotensin II Receptor, Type I, Apolipoprotein E Genotyping, Argininosuccinicaciduria, Aspartylglycosaminuria, Ataxia with Vitamin E Deficiency, Ataxia-Telangiectasia, Autoimmune Polyendocrinopathy Syndrome Type 1, BRCA1 Hereditary Breast/Ovarian Cancer, BRCA2 Hereditary Breast/Ovarian Cancer, Bardet-Biedl Syndrome, Best Vitelliform Macular Dystrophy, Beta-Sarcoglycanopathy, Beta-Thalassemia, Biotinidase Deficiency, Blau Syndrome, Bloom Syndrome, CFTR-Related Disorders, CLN3-Related Neuronal Ceroid-Lipofuscinosis, CLNS-Related Neuronal Ceroid-Lipofuscinosis, CLN8-Related Neuronal Ceroid-Lipofuscinosis, Canavan Disease, Carnitine Palmitoyltransferase IA Deficiency, Carnitine Palmitoyltransferase II Deficiency, Cartilage-Hair Hypoplasia, Cerebral Cavernous Malformation, Choroideremia, Cohen Syndrome, Congenital Cataracts, Facial Dysmorphism, and Neuropathy, Congenital Disorder of Glycosylationla, Congenital Disorder of Glycosylation Ib, Congenital Finnish Nephrosis, Crohn Disease, Cystinosis, DFNA 9 (COCH), Diabetes and Hearing Loss, Early-Onset Primary Dystonia (DYTI), Epidermolysis Bullosa Junctional, Herlitz-Pearson Type, FANCC-Related Fanconi Anemia, FGFR1-Related Craniosynostosis, FGFR2-Related Craniosynostosis, FGFR3-Related Craniosynostosis, Factor V Leiden Thrombophilia, Factor V R2 Mutation Thrombophilia, Factor XI Deficiency, Factor XIII Deficiency, Familial Adenomatous Polyposis, Familial Dysautonomia, Familial Hypercholesterolemia Type B, Familial Mediterranean Fever, Free Sialic Acid Storage Disorders, Frontotemporal Dementia with Parkinsonism-17, Fumarase deficiency, GJB2-Related DFNA 3 Nonsyndromic Hearing Loss and Deafness, GJB2-Related DFNB 1 Nonsyndromic Hearing Loss and Deafness, GNE-Related Myopathies, Galactosemia, Gaucher Disease, Glucose-6-Phosphate Dehydrogenase Deficiency, Glutaricacidemia Type 1, Glycogen Storage Disease Type la, Glycogen Storage Disease Type Ib, Glycogen Storage Disease Type II, Glycogen Storage Disease Type III, Glycogen Storage Disease Type V, Gracile Syndrome, HFE-Associated Hereditary Hemochromatosis, Halder AIMS, Hemoglobin S Beta-Thalassemia, Hereditary Fructose Intolerance, Hereditary Pancreatitis, Hereditary Thymine-Uraciluria, Hexosaminidase A Deficiency, Hidrotic Ectodermal Dysplasia 2, Homocystinuria Caused by Cystathionine Beta-Synthase Deficiency, Hyperkalemic Periodic Paralysis Type 1, Hyperornithinemia-Hyperammonemia-Homocitrullinuria Syndrome, Hyperoxaluria, Primary, Type 1, Hyperoxaluria, Primary, Type 2, Hypochondroplasia, Hypokalemic Periodic Paralysis Type 1, Hypokalemic Periodic Paralysis Type 2, Hypophosphatasia, Infantile Myopathy and Lactic Acidosis (Fatal and Non-Fatal Forms), Isovaleric Acidemias, Krabbe Disease, LGMD2I, Leber Hereditary Optic Neuropathy, Leigh Syndrome, French-Canadian Type, Long Chain 3-Hydroxyacyl-CoA Dehydrogenase Deficiency, MELAS, MERRF, MTHFR Deficiency, MTHFR Thermolabile Variant, MTRNR1-Related Hearing Loss and Deafness, MTTS1-Related Hearing Loss and Deafness, MYH-Associated Polyposis, Maple Syrup Urine Disease Type 1A, Maple Syrup Urine Disease Type 1B, McCune-Albright Syndrome, Medium Chain Acyl-Coenzyme A Dehydrogenase Deficiency, Megalencephalic Leukoencephalopathy with Subcortical Cysts, Metachromatic Leukodystrophy, Mitochondrial Cardiomyopathy, Mitochondrial DNA-Associated Leigh Syndrome and NARP, Mucolipidosis IV, Mucopolysaccharidosis Type I, Mucopolysaccharidosis Type IIIA, Mucopolysaccharidosis Type VII, Multiple Endocrine Neoplasia Type 2, Muscle-Eye-Brain Disease, Nemaline Myopathy, Neurological phenotype, Niemann-Pick Disease Due to Sphingomyelinase Deficiency, Niemann-Pick Disease Type C1, Nijmegen Breakage Syndrome, PPT1-Related Neuronal Ceroid-Lipofuscinosis, PROP1-related pituitary hormone deficiency, Pallister-Hall Syndrome, Paramyotonia Congenita, Pendred Syndrome, Peroxisomal Bifunctional Enzyme Deficiency, Pervasive Developmental Disorders, Phenylalanine Hydroxylase Deficiency, Plasminogen Activator Inhibitor I, Polycystic Kidney Disease, Autosomal Recessive, Prothrombin G20210A Thrombophilia, Pseudovitamin D Deficiency Rickets, Pycnodysostosis, Retinitis Pigmentosa, Autosomal Recessive, Bothnia Type, Rett Syndrome, Rhizomelic Chondrodysplasia Punctata Type 1, Short Chain Acyl-CoA Dehydrogenase Deficiency, Shwachman-Diamond Syndrome, Sjogren-Larsson Syndrome, Smith-Lemli-Opitz Syndrome, Spastic Paraplegia 13, Sulfate Transporter-Related Osteochondrodysplasia, TFR2-Related Hereditary Hemochromatosis, TPP1-Related Neuronal Ceroid-Lipofuscinosis, Thanatophoric Dysplasia, Transthyretin Amyloidosis, Trifunctional Protein Deficiency, Tyrosine Hydroxylase-Deficient DRD, Tyrosinemia Type I, Wilson Disease, X-Linked Juvenile Retinoschisis, cystic fibrosis, spinal muscular atrophy (SMA), a hemoglobinopathy, and Zellweger Syndrome Spectrum.

For the purposes of the disclosed methods, identification or detection of genetic variants can be performed using the in silico moving window analysis to establish a trajectory based on allele balance (when assessing genetic variants involving a SNP, Indel, or other point mutation) across the analyzed windows, as described herein, or based on depth (when assessing genetic variants involving a copy number change). This analysis may be particularly useful for detecting recessive conditions, traits, or diseases. As described above, this process can comprise a read-length-based size exclusion of sequences in at least two windows of the sequence library, thereby obtaining at least two fetal fraction-enriched sequence libraries. Comparing the allele balance in each window allows for the calculation of an allele balance trajectory between the various fetal-fraction enriched sequence libraries. The allele balance trajectory is the change across the observed windows of the percentage of the allele balance for any given genetic sequence of interest. The allele balance trajectory can be calculated as a slope of the allele balance versus fetal fraction across observed window, and it can be visualized in a number of ways, as shown in FIG. 3.

Allele balance trajectories can be utilized to identify heterozygous and homozygous mutations within the cfDNA library. For example, a single point in the trajectory is based on the allele balance in a given window and could be converted to a banding pattern plot in which the y-axis displays the percentage of cfDNA in a sample with a given allele for a particular gene or nucleic acid sequence of interest (e.g., 0%, 10%, 20%, 30%, 40%, 50%, 60%), and the x-axis displays different alleles (i.e., reference allele or alt allele) for the gene or nucleic acid of interest that correspond to the wild-type sequence or a mutation/variant associated with a particular disease, condition, or trait (e.g., different known mutations within the CFTR gene that are associated with cystic fibrosis). If the fetal fraction in the window or sample was, for example, 20%, then a band at 10% on the y-axis corresponds to a fetus that is a carrier from the biological father's DNA or a de novo mutation in the fetus. A band at 40% on the y-axis corresponds to a fetus that is negative (i.e., homozygous reference) for the mutation/variant in the gene or sequence of interest and the mother is heterozygous (i.e., a carrier). A band at 50% on the y-axis corresponds to a fetus that is a carrier from the biological mother's DNA or, in instances in which the mother are father are both carriers with the same alt allele, a carrier of the biological father's DNA. A band at 60% on the y-axis corresponds to a fetus that is homozygous positive for the mutation/variant in the gene or sequence of interest. As noted above, the bands discussed above (i.e., at 10%, 40%, 50%, and 60%) are not fixed and their position will vary based on the fetal fraction. For example, if the fetal fraction were instead 10% (as opposed to 20% in the example above), the values of the bands change from 10%, 40%, 50%, and 60% to 5%, 45%, 50%, and 55%, respectively.

An allele balance trajectory incorporates this static information from each of the observed window, which will necessarily have different fetal fractions. Thus, a trajectory could rely on a window with the 20% fetal fraction described above, a second window with a 10% fetal fraction, and, optionally, 1, 2, 3, 4, 5, 6, 7, 8, 9, or 10 more windows with varying fetal fractions. p Additionally or alternatively, specific callers for genetic variants of interest can rely on assessment of copy number, depth analysis (as described above with respect to aneuploidy), or other forms of detection known in the art. For example, depth trajectories can be used to detect the presence or absence of copy number variants, such as copy number variants of SMA1, RHD, HBA1 and HBA 2, which are all associated with particular genetic diseases. In some embodiments, a depth trajectory may have a negative slope (indicating fewer copies in the fetus), an approximately flat slope (indicating the same number of copies between the fetus and mother), or a positive slope (indicating more copies in the fetus, and such slopes may be based on 1, 2, 3, 4, 5, 6, 7, 8, 9, or 10 more windows with varying fetal fractions.

In some embodiments, callers for certain conditions may rely on detecting the presence or absence of “diffbases.” In some embodiments, callers for certain conditions may rely on detecting the presence or absence of substitutions from a wild type sequence (e.g., SNVs). In some embodiments, callers for certain conditions may rely on detecting the presence or absence of single nucleotide polymorphisms (SNPs). In some embodiments, callers for certain conditions may rely on detecting the presence or absence of one or more insertions or deletions (INDELs). In instances when multiple SNVs, diffbases, SNPs, or a combination thereof are associated with a given condition, pooling or merging detection signals across even a small number of SNVs, diffbases, SNPs, INDELs, or a combination thereof (e.g., <3, <4, <5, <6, <7, <8, <9, <10, <11, <12, <13, <14, <15) can provide improved separation between genotypes. Accordingly, in some embodiments, a caller for a certain condition may rely on the detection of the presence or absence of 1, 2, 3, 4, 5, 6, 7, 8, 9, 10, 11, 12, 13, 14, or 15 or more SNVs, diffbases, SNPs, INDELs, or combinations thereof.

For example, in detecting alpha thalassemia, the disclosed methods can utilize a caller that detects the presence or absence of double cis mutations of HBA1 and HBA2, which is the most common cause of the condition. Thus, a caller may detect, for example, a consensus copy number signal obtained from multiple probes in a region of interest, such as a double deletion region.

As such, utilization of the disclosed methods allows for calling genetic variants of interest with a single sample and in parallel with detection of aneuploidy. Indeed, this method can even identify whether the fetus is a homozygous or heterozygous for a given genetic variant of interest. Further, in some embodiments in which a mother and father possess different alt alleles, it can be determined whether the fetus obtained a particular variant from the mother, the father, or both. This is a new and useful way to establish the presence of absence of a genetic variant/mutation from a maternal sample.

D. Reduction of Noise

As explained above and further shown in the examples, the disclosed methods and systems can significantly reduce noise in cfDNA data, which improves performance of assays used to detect genetic variants and aneuploidies. Due to low levels of cffDNA in most biological samples obtained from pregnant women, high levels of background noise from conventional processing and detection methods could render a sample unusable, uninterpretable, or both. Accordingly, the disclosed methods of noise reduction represent new and useful methods that improve conventional non-invasive pre-natal screening (NIPS).

Additionally, the present disclosure provides methods of reducing background noise from superfluous genetic material in non-invasive pre-natal screening (NIPS), comprising (i) obtaining a biological sample from a pregnant woman, wherein the biological sample comprises cell-free DNA (cfDNA); and (ii) processing the cfDNA used for NIPS, wherein processing comprises enriching the biological sample for cell-free fetal DNA (cffDNA), in silico processing of the cfDNA, or a combination thereof.

In some embodiments, the methods of reducing noise will comprise both enriching the biological sample for cell-free fetal DNA (cffDNA) and in silico processing of the cfDNA.

For the purposes of reducing noise, the enriching of the biological sample for cffDNA may comprise any of the disclosed methods of physical isolation or enrichment of a fetal fraction. For example, in some embodiments, enriching the biological sample for cffDNA may comprise obtaining a biological sample comprising cell-free DNA (cfDNA) from a pregnant woman, wherein the cfDNA comprises cffDNA and cell-free DNA maternal (cfmDNA); extracting the cfDNA from the biological sample; and subjecting the extracted cfDNA to a size exclusion process, wherein the size exclusion process has a cutoff size of about 150 nucleotides in length, about 155 nucleotides in length, about 160 nucleotides in lengths, about 165 nucleotides in length, about 170 nucleotides in length, about 175 nucleotides in length, or about 180 nucleotides in length, thereby producing a nucleic acid enriched for cffDNA.

Similarly, for the purposes of reducing noise, the in silico processing may comprise any of the disclosed methods of analysis of sequence libraries or sequence library data to focus any analysis of genetic variants or aneuploidies on the fetal fraction of a sample. For example, in some embodiments, in silico processing may comprise sequencing a cfDNA sample comprising cell-free fetal (cffDNA) and cell-free maternal DNA (cfmDNA) to prepare a sequence library; performing read-length-based analysis in which an allele balance for a nucleic acid sequence of interest is established in at least two windows of the sequence library; and establishing a trajectory based on the allele balance of the at least two windows, wherein the trajectory indicates the percentage of alleles present in the sample that comprise the nucleic acid sequence of interest.

Noise reduction may further comprise normalization to control for GC-bias, sample background, hybridization probe capture, or a combination thereof In general, normalization can be a “median normalization.” In other words, probe read depths can be divided by the median across probes with similar GC content, then by the interquartile mean across samples and probes with putative copy number 2 in mother and fetus.

Issues may arise with hybridization probe capture because DNA fragments that contain variants and overlap capture probes are captured less efficiently, decreasing allele balance of the alternate allele. However, capture bias is often reproducible, and in such cases it can be learned and corrected using the following formula:

$$Bias = \frac{1 - {AB}_{expected}}{{AB}_{expected}} \cdot \frac{{AB}_{observed}}{1 - {AB}_{observed}} \qquad [ Formula 2 ]$$

Correction and normalization for hybridization probe capture is particularly useful for ensuring correct indel calling, though it can help with variant calling more generally.

The following examples are given to illustrate the disclosed sample preparations and methods. It should be understood, however, that the invention is not to be limited to the specific embodiments or details described in these examples.

### EXAMPLES

### Example 1—Aneuploidy Caller Performance

The disclosed aneuploidy caller is based on sequence read depth as described above. To establish feasibility of this approach, 110 feasibility samples were analyzed and compared against a standard accepted aneuploidy detection system (Myriad PREQUEL™ Prenatal Screen). The aneuploidies detectable in the samples and the control call for each is shown in the table below:

| | Prequel Call | samples |
|---|---|---|
| Autosomal | Trisomy 13 | 9 |
| | Trisomy 18 | 10 |
| | Trisomy 21 | 10 |
| | 22q microdel | 5 |
| SCA | Monosomy X | 2 |
| | Trisomy X | 3 |
| | XXY | 2 |
| | XYY | 1 |
| | Negative | 68 |

The disclosed depth-based method of analysis provided the following results:

For Autosomal+22q
Sensitivity=100% (CI: 89.95-100%)
Specificity=99.75% (CI: 98.59-99.96%)
One false positive mosaic monosomy 21 call

For Sex Chromosome Aneuploidy
Sensitivity=100% (CI: 63.06-100%)
Specificity=100% (CI: 96.41-100%)

For Fetal Sex Calls
100% concordance with control test

Only one sample of the 110 samples failed for low depth (0.9% re-run rate).

### Example 2—SNV/Indel (i.e., Genetic Variant) Caller Performance

Fifteen (15) contrived mixtures from 5 prenatal pairs were used to validate the performance of the SNV/indel caller system disclosed herein. The sensitivity and specificity for a gene region of interest (ROI), alone and in combination with a set of SNVs known to have high variability within the population (i.e., dbSNP) is shown in the table below:

| | | | | Gene ROI + | | CFTR |
|---|---|---|---|---|---|---|
| | | Gene ROI | Gene ROI | dbSNP | Gene ROI + dbSNP | ΔF508 |
| | | sensitivity | specificity | sensitivity | specificity | accuracy |
| | | (95% CI) | (95% CI) | (95% CI) | (95% CI) | (n = 6) |
| Fetal | Paternal | 100% | 98.0% | 99.9% | 99.99% | NA |
| genotyping | inherited | (95.55%-100%) | (95.41%-99.35%) | (99.79%-99.92%) | (99.97%-100%) | |
| assignment | Maternal | 100% | 98.9% | 98.7% | 98.9% | 100% |
| | inherited | (97.89%-100%) | (97.41%-99.64%) | (98.53%-98.73%) | (98.85%-99.02%) | |
| Maternal | | 100% | 100% | 99.99% | 99.997% | 100% |
| carrier | | (98.61%-100%) | (99.30%-100%) | (99.98%-99.99%) | (99.992%-99.999%) | |
| status | | | | | | |
| assignment | | | | | | |

This initial performance was established without the use of the physical enrichment processes described herein. It is expected that enriching the fetal fraction and optimizing variant filter parameters would further improve performance.

Additionally, the disclosed single gene SNV/indel caller met performance requirements on 5 unique cfDNA samples with a FF of 5.8-16%. The results are shown in the table below.

| | | | | Gene ROI + | | CFTR |
|---|---|---|---|---|---|---|
| | | Gene ROI | Gene ROI | dbSNP | Gene ROI + dbSNP | ΔF508 |
| | | sensitivity | specificity | sensitivity | specificity | accuracy |
| | | (95% CI) | (95% CI) | (95% CI) | (95% CI) | (n = 6) |
| Fetal | Paternal | 100% | 99.1% | 99.6% | 99.99% | NA |
| genotyping | inherited | (92.13%-100%) | (94.95%-99.98%) | (99.45%-99.76%) | (99.96%-100%) | |
| assignment | Maternal | 100% | 99.4% | 98.5% | 98.96% | 100% |
| | inherited | (94.87%-100%) | (96.65%-99.98%) | (98.35%-98.66%) | (98.82%-99.07%) | |
| Maternal | | 100% | 100% | 99.99% | 100% | 100% |
| carrier | | (96.82%-100%) | (98.05%-100%) | (99.97%-100%) | (99.99%-100%) | |
| status | | | | | | |
| assignment | | | | | | |

For this performance assessment, the best performance was observed when both physical size-exclusion based enrichment and bias corrections were both implemented.

### Example 3—SMA Caller Performance Analysis

Spinal muscular atrophy (SMA) is a genetically inheritable condition that is commonly included in prenatal screenings. However, SMA calling is difficult due to the high degree of homology between the SMN1 and SMN2 genes. These genes differ at very few positions (most notably exon 7), and SMA carrier/affected status depends only on SMN1 copy number.

The disclosed system assessed the presence or absence of multiple bases (up to 44 diffbases) to ensure correct calling. As shown in the table below, the SMA caller was highly accurate, sensitive, and specific.

| | # | | | | | | |
|---|---|---|---|---|---|---|---|
| | Samples | | | | | | |
| | (carrier | | | | | | |
| | moms; | | | | | | |
| Type of | affected | Maternal carrier status | | | Fetal Status (Affected vs. Healthy) | | |
| Sample | fetuses) | Accuracy | Sensitivity | Specificity | Accuracy | Sensitivity | Specificity |
| Coriell | 30 | 100% | 100% | 100% | 100% | 100% | 100% |
| contrived | (6; 6) | (88.4-100%) | (54.1-100%) | (85.8-100%) | (88.4-100%) | (54.1-100%) | (85.8-100%) |
| mix | | | | | | | |
| Internal | 10 | 100% | N/A | 100% | 100%** | N/A | 100% |
| Plasma | (0; 0) | (69.1-100%) | | (69.1-100%) | (69.1-100%) | | (69.1-100%) |
| External | 5 | 100% | N/A | 100% | 100% | N/A | 100% |
| Plasma | (0; 0) | (47.8-100%) | | (47.8-100%) | (47.8-100%) | | (47.8-100%) |

For the purposes of this assessment, carrier fetuses were considered healthy.

### Example 4—Alpha Thalassemia Caller Performance Analysis

Alpha thalassemia is a blood disorder that reduces the production of hemoglobin. It is a genetically inheritable condition that is commonly included in prenatal screenings. The disclosed system assessed the presence or absence of double cis mutations of HBA1 and HBA2, which is the most common cause of the condition. More specifically, a consensus copy number signal was obtained from multiple probes in the double deletion region. As shown in the table below, the Alpha Thalassemia caller was highly accurate, sensitive, and specific.

| | # | | | | | | |
|---|---|---|---|---|---|---|---|
| | Samples | | | | | | |
| | (carrier | | | | | | |
| | moms; | | | | | | |
| Type of | affected | Maternal carrier status | | | Fetal Status (Affected vs. Healthy) | | |
| Sample | fetuses) | Accuracy | Sensitivity | Specificity | Accuracy | Sensitivity | Specificity |
| Coriell | 19 | 100% | 100% | 100% | 100% | 100% | 100% |
| contrived | (3; 3) | (82.4-100%) | (29.2-100%) | (79.4-100%) | (82.4-100%) | (29.2-100%) | (79.4-100%) |
| mix | | | | | | | |
| Internal | 10 | 100% | 100% | 100% | 100% | 100% | 100% |
| Plasma | (4; 1) | (69.1-100%) | (47.8-100%) | (47.8-100%) | (69.1-100%) | (39.8-100%) | (54.1-100%) |
| External | 5 | 100% | N/A | 100% | 100% | N/A | 100% |
| Plasma | (0; 0) | (47.8-100%) | | (47.8-100%) | (47.8-100%) | | (47.8-100%) |

For the purposes of this assessment, carrier fetuses were considered healthy.

### Example 5—RhD Caller Performance Analysis

If a pregnant mother is D(−) and the fetus is D(+) a hemolytic disease can occur when maternal blood is exposed to fetal blood. This condition is commonly included in prenatal screenings. The most common cause of RhD(−) is a whole gene deletion of RHD. Thus, a caller was developed based on 221 reliable diffbases to assess copy number. As shown in the table below, the RhD caller was highly accurate, sensitive, and specific.

| | # | | | | | | |
|---|---|---|---|---|---|---|---|
| | Samples | | | | | | |
| | (RHD- | | | | | | |
| | moms; | | | | | | |
| | RHD- | | | | | | |
| | moms | | | | | | |
| | with | | | | | | |
| Type of | RHD + | Maternal RhD status | | | RhD_ Fetus with RhD-Mother | | |
| Sample | fetuses) | Accuracy | Sensitivity | Specificity | Accuracy | Sensitivity | Specificity |
| Coriell | 19 | 100% | 100% | 100% | 100% | 100% | N/A |
| contrived | (7; 7) | (82.4-100%) | (59.0-100%) | (73.5-100%) | (59.0-100%) | (59.0-100%) | |
| mix | | | | | | | |
| External | 5 | 100% | N/A | 100% | 100% | N/A | 100% |
| Plasma | (0; 0) | (47.8-100%) | | (47.8-100%) | (47.8-100%) | | (47.8-100%) |

All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds, compositions, or systems, which can, of course, vary. It is also to be understood that the terminology used herein is for the purpose of describing particular embodiments only, and is not intended to be limiting.

1. A method of preparing a biological sample with an enriched fetal fraction, comprising:
(a-1) obtaining a biological sample comprising cell-free DNA (cfDNA) from a pregnant woman;
(b-1) extracting cfDNA from the biological sample;
(c-1) preparing a library of cfDNA fragments to obtain a cfDNA library
(d-1) separating the cfDNA fragments in the cfDNA library by size to retain only cfDNA fragments that are less than about 150 nucleotides in length, about 155 nucleotides in length, about 160 nucleotides in lengths, about 165 nucleotides in length, about 170 nucleotides in length, about 175 nucleotides in length, about 180 nucleotides in length, about 185 nucleotides in length, about 190 nucleotides in length, about 195 nucleotides in length, or about 200 nucleotides in length;
(e-1) sequencing the retained cfDNA fragments to obtain a first sequence library;
(f-1) identify based on read-length length (i) sequences of cell-free fetal DNA (cffDNA) and (ii) sequences of cell-free maternal DNA (cfmDNA) that are present in at least two windows of the first sequence library; and
(g-1) isolating the sequences of cffDNA from each of the at least two windows of the sequence library, thereby obtaining at least two fetal fraction-enriched sequence libraries or
(a-2) obtaining a biological sample comprising cell-free DNA (cfDNA) from a pregnant woman;
(b-2) extracting cfDNA from the biological sample;
(c-2) separating cfDNA fragments in the extracted sample from (b-2) to retain only cfDNA fragments that are less than about 150 nucleotides in length, about 155 nucleotides in length, about 160 nucleotides in lengths, about 165 nucleotides in length, about 170 nucleotides in length, about 175 nucleotides in length, about 180 nucleotides in length, about 185 nucleotides in length, about 190 nucleotides in length, about 195 nucleotides in length, or about 200 nucleotides in length;
(d-2) preparing a cfDNA library from the separated cfDNA fragments from (c-2);
(e-2) sequencing the cfDNA library to obtain a first sequence library;
(f-2) identify based on read-length length (i) sequences of cell-free fetal DNA (cffDNA) and (ii) sequences of cell-free maternal DNA (cfmDNA) that are present in at least two windows of the first sequence library; and
(g-2) isolating the sequences of cffDNA from each of the at least two windows of the sequence library, thereby obtaining at least two fetal fraction-enriched sequence libraries.

2. The method of claim 1, wherein separating the cfDNA fragments enriches the fetal fraction in the biological sample by about 1.1 fold, about 1.2 fold, about 1.3 fold, about 1.4 fold, about 1.5 fold, about 1.6 fold, about 1.7 fold, about 1.8 fold, about 1.9 fold, or about 2.0 fold.

3. The method of claim 1, wherein isolating the sequences of cffDNA from the at least two windows of the first sequence library enriches the fetal fraction in the biological sample by about 1.1 fold, about 1.2 fold, about 1.3 fold, about 1.4 fold, about 1.5 fold, about 1.6 fold, about 1.7 fold, about 1.8 fold, about 1.9 fold, about 2.0 fold, about 2.1 fold, about 2.2 fold, about 2.3 fold, about 2.4 fold, about 2.5 fold, about 2.6 fold, about 2.7 fold, about 2.8 fold, about 2.9 fold, about 3.0 fold, about 3.1 fold, about 3.2 fold, about 3.3 fold, about 3.4 fold, or about 3.5 fold.

4. The method of claim 1, wherein separating the cfDNA fragments comprises electrophoresis.

5. The method of claim 1, wherein at least 3, at least 4, at least 5, at least 6, at least 7, at least 8, at least 9, or at least 10 windows of the first sequence library are assessed to identify and isolate cffDNA sequences, thereby obtaining, respectively, at least 3, at least 4, at least 5, at least 6, at least 7, at least 8, at least 9, or at least 10 fetal fraction-enriched sequence libraries.

6. The method of claim 1 further comprises identifying and separating cffDNA from cfmDNA by comparing sequence reads of cffDNA and cfmDNA in the first sequence library to a reference genome, demultiplexing sequence reads from the first library, removing duplicate sequences from the first sequence library, or a combination thereof.

7. The method of claim 1 further comprising assessing the at least two fetal fraction-enriched sequence libraries for the presence of one or more genetic mutation(s).

8. The method of claim 7, wherein the one or more genetic mutation(s) cause at least one condition selected from 21-Hydroxylase Deficiency, ABCC8-Related Hyperinsulinism, ARSACS, Achondroplasia, Achromatopsia, Adenosine Monophosphate Deaminase 1, Agenesis of Corpus Callosum with Neuronopathy, Alkaptonuria, Alpha-1-Antitrypsin Deficiency, Alpha-Mannosidosis, Alpha-Sarcoglycanopathy, Alpha-Thalassemia, Alzheimers, Angiotensin II Receptor, Type I, Apolipoprotein E Genotyping, Argininosuccinicaciduria, Aspartylglycosaminuria, Ataxia with Vitamin E Deficiency, Ataxia-Telangiectasia, Autoimmune Polyendocrinopathy Syndrome Type 1, BRCA1 Hereditary Breast/Ovarian Cancer, BRCA2 Hereditary Breast/Ovarian Cancer, Bardet-Biedl Syndrome, Best Vitelliform Macular Dystrophy, Beta-Sarcoglycanopathy, Beta-Thalassemia, Biotinidase Deficiency, Blau Syndrome, Bloom Syndrome, CFTR-Related Disorders, CLN3-Related Neuronal Ceroid-Lipofuscinosis, CLN5-Related Neuronal Ceroid-Lipofuscinosis, CLN8-Related Neuronal Ceroid-Lipofuscinosis, Canavan Disease, Carnitine Palmitoyltransferase IA Deficiency, Carnitine Palmitoyltransferase II Deficiency, Cartilage-Hair Hypoplasia, Cerebral Cavernous Malformation, Choroideremia, Cohen Syndrome, Congenital Cataracts, Facial Dysmorphism, and Neuropathy, Congenital Disorder of Glycosylationla, Congenital Disorder of Glycosylation Ib, Congenital Finnish Nephrosis, Crohn Disease, Cystinosis, DFNA 9 (COCH), Diabetes and Hearing Loss, Early-Onset Primary Dystonia (DYTI), Epidermolysis Bullosa Junctional, Herlitz-Pearson Type, FANCC-Related Fanconi Anemia, FGFR1-Related Craniosynostosis, FGFR2-Related Craniosynostosis, FGFR3-Related Craniosynostosis, Factor V Leiden Thrombophilia, Factor V R2 Mutation Thrombophilia, Factor XI Deficiency, Factor XIII Deficiency, Familial Adenomatous Polyposis, Familial Dysautonomia, Familial Hypercholesterolemia Type B, Familial Mediterranean Fever, Free Sialic Acid Storage Disorders, Frontotemporal Dementia with Parkinsonism-17, Fumarase deficiency, GJB2-Related DFNA 3 Nonsyndromic Hearing Loss and Deafness, GJB2-Related DFNB 1 Nonsyndromic Hearing Loss and Deafness, GNE-Related Myopathies, Galactosemia, Gaucher Disease, Glucose-6-Phosphate Dehydrogenase Deficiency, Glutaricacidemia Type 1, Glycogen Storage Disease Type la, Glycogen Storage Disease Type Ib, Glycogen Storage Disease Type II, Glycogen Storage Disease Type III, Glycogen Storage Disease Type V, Gracile Syndrome, HFE-Associated Hereditary Hemochromatosis, Halder AIMS, Hemoglobin S Beta-Thalassemia, Hereditary Fructose Intolerance, Hereditary Pancreatitis, Hereditary Thymine-Uraciluria, Hexosaminidase A Deficiency, Hidrotic Ectodermal Dysplasia 2, Homocystinuria Caused by Cystathionine Beta-Synthase Deficiency, Hyperkalemic Periodic Paralysis Type 1, Hyperornithinemia-Hyperammonemia-Homocitrullinuria Syndrome, Hyperoxaluria, Primary, Type 1, Hyperoxaluria, Primary, Type 2, Hypochondroplasia, Hypokalemic Periodic Paralysis Type 1, Hypokalemic Periodic Paralysis Type 2, Hypophosphatasia, Infantile Myopathy and Lactic Acidosis (Fatal and Non-Fatal Forms), Isovaleric Acidemias, Krabbe Disease, LGMD2I, Leber Hereditary Optic Neuropathy, Leigh Syndrome, French-Canadian Type, Long Chain 3-Hydroxyacyl-CoA Dehydrogenase Deficiency, MELAS, MERRF, MTHFR Deficiency, MTHFR Thermolabile Variant, MTRNR1-Related Hearing Loss and Deafness, MTTS1-Related Hearing Loss and Deafness, MYH-Associated Polyposis, Maple Syrup Urine Disease Type 1A, Maple Syrup Urine Disease Type 1B, McCune-Albright Syndrome, Medium Chain Acyl-Coenzyme A Dehydrogenase Deficiency, Megalencephalic Leukoencephalopathy with Subcortical Cysts, Metachromatic Leukodystrophy, Mitochondrial Cardiomyopathy, Mitochondrial DNA-Associated Leigh Syndrome and NARP, Mucolipidosis IV, Mucopolysaccharidosis Type I, Mucopolysaccharidosis Type IIIA, Mucopolysaccharidosis Type VII, Multiple Endocrine Neoplasia Type 2, Muscle-Eye-Brain Disease, Nemaline Myopathy, Neurological phenotype, Niemann-Pick Disease Due to Sphingomyelinase Deficiency, Niemann-Pick Disease Type C1, Nijmegen Breakage Syndrome, PPT1-Related Neuronal Ceroid-Lipofuscinosis, PROP1-related pituitary hormone deficiency, Pallister-Hall Syndrome, Paramyotonia Congenita, Pendred Syndrome, Peroxisomal Bifunctional Enzyme Deficiency, Pervasive Developmental Disorders, Phenylalanine Hydroxylase Deficiency, Plasminogen Activator Inhibitor I, Polycystic Kidney Disease, Autosomal Recessive, Prothrombin G20210A Thrombophilia, Pseudovitamin D Deficiency Rickets, Pycnodysostosis, Retinitis Pigmentosa, Autosomal Recessive, Bothnia Type, Rett Syndrome, Rhizomelic Chondrodysplasia Punctata Type 1, Short Chain Acyl-CoA Dehydrogenase Deficiency, Shwachman-Diamond Syndrome, Sjogren-Larsson Syndrome, Smith-Lemli-Opitz Syndrome, Spastic Paraplegia 13, Sulfate Transporter-Related Osteochondrodysplasia, TFR2-Related Hereditary Hemochromatosis, TPP1-Related Neuronal Ceroid-Lipofuscinosis, Thanatophoric Dysplasia, Transthyretin Amyloidosis, Trifunctional Protein Deficiency, Tyrosine Hydroxylase-Deficient DRD, Tyrosinemia Type I, Wilson Disease, X-Linked Juvenile Retinoschisis, cystic fibrosis, spinal muscular atrophy (SMA), a hemoglobinopathy, and Zellweger Syndrome Spectrum.

9. The method of claim 1 further comprising assessing the biological sample comprising cfDNA for the presence of an aneuploidy.

10. The method of claim 9, wherein the aneuploidy is selected from a monosomy, a trisomy, a tetrasomy, a pentasomy, a microdeletion, a micoduplication, and mosaic versions of monosomy, trisomy, tetrasomy, and pentasomy.

11. A method of parallel detecting the presence or absence of aneuploidy and the presence or absence of at least one genetic variant in a single, maternal sample, comprising
(i) obtaining a biological sample from a pregnant woman, wherein the biological sample comprises cell-free DNA (cfDNA);
(ii) preparing a cfDNA library;
(iii) sequencing the cfDNA library to produce a sequence library; and
(iv) detecting the presence or absence of aneuploidy and the presence or absence of at least one genetic variant in the single, maternal sample;
wherein (a) the cfDNA library is enriched to increase a fetal fraction, (b) the sequence library is enriched to increase a fetal fraction, or (c) a combination thereof, such that the fetal fraction of the single maternal sample is increased at least 1.1. fold, at least 1.2 fold, at least 1.3 fold, at least 1.4 fold, or at least 1.5 fold prior to detecting the presence or absence of aneuploidy and the presence or absence of at least one genetic variant in the single, maternal sample.

12. The method of claim 11, wherein the biological sample is blood or plasma.

13. The method of claim 11, wherein the cfDNA library is enriched to increase the fetal fraction and the sequence library is enriched to increase the fetal fraction.

14. The method of claim 11, wherein enriching the fetal fraction of the cfDNA library comprises removing from the cfDNA library any DNA fragments that are greater than about 150 nucleotides in length, about 155 nucleotides in length, about 160 nucleotides in lengths, about 165 nucleotides in length, about 170 nucleotides in length, about 175 nucleotides in length, or about 180 nucleotides in length.

15. The method of claim 14, wherein removing the DNA fragments from the cfDNA library comprises electrophoresis.

16. The method of claim 11, wherein enriching the fetal fraction of the sequence library comprises a read-length-based size exclusion of sequences in at least two windows of the sequence library, thereby obtaining at least two fetal fraction-enriched sequence libraries.

17. The method of claim 16, wherein at least 3, at least 4, at least 5, at least 6, at least 7, at least 8, at least 9, or at least 10 windows of the first sequence library are assessed to identify and isolate cffDNA sequences, thereby obtaining, respectively, at least 3, at least 4, at least 5, at least 6, at least 7, at least 8, at least 9, or at least 10 fetal fraction-enriched sequence libraries.

18. The method of claim 16, wherein the at least two windows of the sequence library are selected from (i) sequences that are 0-145 nucleotides, (ii) sequences that are 0-150 nucleotides, (iii) 0-155 nucleotides, (iv) 0-160 nucleotides, (v) 0-165 nucleotides, (vi) 0-168 nucleotides, (vii) 0-170 nucleotides, (viii) 0-175 nucleotides, (ix) 0-180 nucleotides, (x) 0-185 nucleotides, (xi) 0-190 nucleotides, (xii) 0-195 nucleotides, (xiii) 0-200 nucleotides, and (xiv) ungated.

19. The method of claim 16, wherein enriching the fetal fraction of the sequence library further comprises identifying and separating cffDNA from cfmDNA by comparing sequence reads of cffDNA and cfmDNA in the first sequence library to a reference genome, demultiplexing sequence reads from the first library, removing duplicate sequences from the first sequence library, or a combination thereof.

20. The method of claim 11, wherein detecting the presence or absence of at least one genetic variant comprises determining in each of the at least two fetal fraction-enriched sequence libraries an allele balance for each allele in the sample that encodes the at least one genetic variant, and generating an allele balance trajectory for each allele based on the allele balance in each of the at least two fetal fraction-enriched sequence libraries, a depth trajectory based on the depth of the at least two fetal fraction-enriched sequence libraries, or a combination of an allele balance trajectory and a depth trajectory.

21. The method of claim 11, wherein detecting the presence or absence of aneuploidy comprises analyzing a sequence depth of at least one sequence corresponding to a chromosome of interest in the sequence library.

22. The method of claim 21, wherein the sequence depth of the at least one sequence corresponding to the chromosome of interest is fit to a model of expected depth for the chromosome of interest.

23. The method of claim 21, wherein the sequence depth is calculated with the formula:
$${d}_{p} = ( 1 - f ) {c}_{m} \frac{{d}_{b}}{2} + {fc}_{f} \frac{{d}_{b}}{2}$$
where:
dₚ is pregnancy depth
f is fetal fraction
cₘ is maternal copy number
d_{b} is background depth
c_{f} is fetal copy number.

24. The method of claim 11, wherein the sequence depth is normalized to control for GC-bias, sample background, hybridization probe capture, or a combination thereof

25. The method of claim 11, wherein the method comprises detecting the presence or absence of aneuploidy selected from a monosomy, a trisomy, a tetrasomy, a polysomy X, a polysomy Y, a microdeletion, a microduplication, a pentasomy, and a combination thereof.

26. The method of claim 11, wherein the at least one genetic variant is associated with a disease selected from 21-Hydroxylase Deficiency, ABCC8-Related Hyperinsulinism, ARSACS, Achondroplasia, Achromatopsia, Adenosine Monophosphate Deaminase 1, Agenesis of Corpus Callosum with Neuronopathy, Alkaptonuria, Alpha-1-Antitrypsin Deficiency, Alpha-Mannosidosis, Alpha-Sarcoglycanopathy, Alpha-Thalassemia, Alzheimers, Angiotensin II Receptor, Type I, Apolipoprotein E Genotyping, Argininosuccinicaciduria, Aspartylglycosaminuria, Ataxia with Vitamin E Deficiency, Ataxia-Telangiectasia, Autoimmune Polyendocrinopathy Syndrome Type 1, BRCA1 Hereditary Breast/Ovarian Cancer, BRCA2 Hereditary Breast/Ovarian Cancer, Bardet-Biedl Syndrome, Best Vitelliform Macular Dystrophy, Beta-Sarcoglycanopathy, Beta-Thalassemia, Biotinidase Deficiency, Blau Syndrome, Bloom Syndrome, CFTR-Related Disorders, CLN3-Related Neuronal Ceroid-Lipofuscinosis, CLN5-Related Neuronal Ceroid-Lipofuscinosis, CLN8-Related Neuronal Ceroid-Lipofuscinosis, Canavan Disease, Carnitine Palmitoyltransferase IA Deficiency, Carnitine Palmitoyltransferase II Deficiency, Cartilage-Hair Hypoplasia, Cerebral Cavernous Malformation, Choroideremia, Cohen Syndrome, Congenital Cataracts, Facial Dysmorphism, and Neuropathy, Congenital Disorder of Glycosylationla, Congenital Disorder of Glycosylation Ib, Congenital Finnish Nephrosis, Crohn Disease, Cystinosis, DFNA 9 (COCH), Diabetes and Hearing Loss, Early-Onset Primary Dystonia (DYTI), Epidermolysis Bullosa Junctional, Herlitz-Pearson Type, FANCC-Related Fanconi Anemia, FGFR1-Related Craniosynostosis, FGFR2-Related Craniosynostosis, FGFR3-Related Craniosynostosis, Factor V Leiden Thrombophilia, Factor V R2 Mutation Thrombophilia, Factor XI Deficiency, Factor XIII Deficiency, Familial Adenomatous Polyposis, Familial Dysautonomia, Familial Hypercholesterolemia Type B, Familial Mediterranean Fever, Free Sialic Acid Storage Disorders, Frontotemporal Dementia with Parkinsonism-17, Fumarase deficiency, GJB2-Related DFNA 3 Nonsyndromic Hearing Loss and Deafness, GJB2-Related DFNB 1 Nonsyndromic Hearing Loss and Deafness, GNE-Related Myopathies, Galactosemia, Gaucher Disease, Glucose-6-Phosphate Dehydrogenase Deficiency, Glutaricacidemia Type 1, Glycogen Storage Disease Type la, Glycogen Storage Disease Type Ib, Glycogen Storage Disease Type II, Glycogen Storage Disease Type III, Glycogen Storage Disease Type V, Gracile Syndrome, HFE-Associated Hereditary Hemochromatosis, Halder AIMS, Hemoglobin S Beta-Thalassemia, Hereditary Fructose Intolerance, Hereditary Pancreatitis, Hereditary Thymine-Uraciluria, Hexosaminidase A Deficiency, Hidrotic Ectodermal Dysplasia 2, Homocystinuria Caused by Cystathionine Beta-Synthase Deficiency, Hyperkalemic Periodic Paralysis Type 1, Hyperornithinemia-Hyperammonemia-Homocitrullinuria Syndrome, Hyperoxaluria, Primary, Type 1, Hyperoxaluria, Primary, Type 2, Hypochondroplasia, Hypokalemic Periodic Paralysis Type 1, Hypokalemic Periodic Paralysis Type 2, Hypophosphatasia, Infantile Myopathy and Lactic Acidosis (Fatal and Non-Fatal Forms), Isovaleric Acidemias, Krabbe Disease, LGMD2I, Leber Hereditary Optic Neuropathy, Leigh Syndrome, French-Canadian Type, Long Chain 3-Hydroxyacyl-CoA Dehydrogenase Deficiency, MELAS, MERRF, MTHFR Deficiency, MTHFR Thermolabile Variant, MTRNR1-Related Hearing Loss and Deafness, MTTS1-Related Hearing Loss and Deafness, MYH-Associated Polyposis, Maple Syrup Urine Disease Type 1A, Maple Syrup Urine Disease Type 1B, McCune-Albright Syndrome, Medium Chain Acyl-Coenzyme A Dehydrogenase Deficiency, Megalencephalic Leukoencephalopathy with Subcortical Cysts, Metachromatic Leukodystrophy, Mitochondrial Cardiomyopathy, Mitochondrial DNA-Associated Leigh Syndrome and NARP, Mucolipidosis IV, Mucopolysaccharidosis Type I, Mucopolysaccharidosis Type IIIA, Mucopolysaccharidosis Type VII, Multiple Endocrine Neoplasia Type 2, Muscle-Eye-Brain Disease, Nemaline Myopathy, Neurological phenotype, Niemann-Pick Disease Due to Sphingomyelinase Deficiency, Niemann-Pick Disease Type C1, Nijmegen Breakage Syndrome, PPT1-Related Neuronal Ceroid-Lipofuscinosis, PROP1-related pituitary hormone deficiency, Pallister-Hall Syndrome, Paramyotonia Congenita, Pendred Syndrome, Peroxisomal Bifunctional Enzyme Deficiency, Pervasive Developmental Disorders, Phenylalanine Hydroxylase Deficiency, Plasminogen Activator Inhibitor I, Polycystic Kidney Disease, Autosomal Recessive, Prothrombin G20210A Thrombophilia, Pseudovitamin D Deficiency Rickets, Pycnodysostosis, Retinitis Pigmentosa, Autosomal Recessive, Bothnia Type, Rett Syndrome, Rhizomelic Chondrodysplasia Punctata Type 1, Short Chain Acyl-CoA Dehydrogenase Deficiency, Shwachman-Diamond Syndrome, Sjogren-Larsson Syndrome, Smith-Lemli-Opitz Syndrome, Spastic Paraplegia 13, Sulfate Transporter-Related Osteochondrodysplasia, TFR2-Related Hereditary Hemochromatosis, TPP1-Related Neuronal Ceroid-Lipofuscinosis, Thanatophoric Dysplasia, Transthyretin Amyloidosis, Trifunctional Protein Deficiency, Tyrosine Hydroxylase-Deficient DRD, Tyrosinemia Type I, Wilson Disease, X-Linked Juvenile Retinoschisis, cystic fibrosis, spinal muscular atrophy (SMA), a hemoglobinopathy, and Zellweger Syndrome Spectrum.

27. A method of enriching a biological sample for cell-free fetal DNA (cffDNA), comprising obtaining a biological sample comprising cell-free DNA (cfDNA) from a pregnant woman, wherein the cfDNA comprises cffDNA and cell-free maternal DNA (cfmDNA); extracting the cfDNA from the biological sample; and subjecting the extracted cfDNA to a size exclusion process, wherein the size exclusion process has a cutoff size of about 150 nucleotides in length, about 155 nucleotides in length, about 160 nucleotides in lengths, about 165 nucleotides in length, about 170 nucleotides in length, about 175 nucleotides in length, or about 180 nucleotides in length, thereby producing a sample enriched for cffDNA.

28. A method of in silico processing of cell-free DNA (cfDNA), comprising sequencing a cfDNA sample comprising cell-free fetal (cffDNA) and cell-free maternal DNA (cfmDNA) to prepare a sequence library; performing read-length-based analysis in which an allele balance for a nucleic acid sequence of interest is established in at least two windows of the sequence library; and establishing a trajectory based on the allele balance of the at least two windows.

29. A method of reducing background noise from superfluous genetic material in non-invasive pre-natal screening (NIPS), comprising
(i) obtaining a biological sample from a pregnant woman, wherein the biological sample comprises cell-free DNA (cfDNA); and
(ii) processing the cfDNA used for NIPS, wherein processing comprises enriching the biological sample for cell-free fetal DNA (cffDNA), in silico processing of the cfDNA, or a combination thereof.

30. The method of claim 29, wherein processing comprises both enriching the biological sample for cell-free fetal DNA (cffDNA) and in silico processing of the cfDNA.

31. The method of claim 29, wherein the enriching the biological sample for cell-free fetal DNA (cffDNA) comprises the method of claim 27.

32. The method of claim 29, wherein the in silico processing of the cfDNA comprises the method of claim 28.

33. The method of claim 29 further comprising normalization to control for GC-bias, sample background, hybridization probe capture, or a combination thereof.
